(19) United States
(12) Patent Application Publication
(10) Pub. No.: US 2023/0013833 A1
(43) Pub. Date: Jan. 19, 2023

(54) METHOD OF CREATING ZERO-BURDEN DIGITAL BIOMARKERS FOR AUTISM, AND EXPLOITING CO-MORBIDITY PATTERNS TO DRIVE EARLY INTERVENTION

(71) Applicant: THE UNIVERSITY OF CHICAGO, Chicago, IL (US)

(72) Inventors: Ishanu CHATTOPADHYAY, Chicago, IL (US); Dmytro ONISHCHENKO, Chicago, IL (US); Yi HUANG, Chicago, IL (US)

(21) Appl. No.: 17/763,089

(22) PCT Filed: Sep. 23, 2020
(86) PCT No.: PCT/US2020/052112

§ 371 Date: Mar. 23, 2022

Related U.S. Application Data
(60) Provisional application No. 62/904,220, filed on Sep. 23, 2019.
(60) Provisional application No. 62/937,604, filed on Nov. 19, 2019.

(51) Int. Cl. G16H 50/20; G06N 7/00; G16H 10/60; G16H 50/30

(52) U.S. Cl. CPC G16H 50/20; G06N 7/005; G16H 10/60; G16H 50/30

(57) ABSTRACT

A diagnosis prediction (DP) computing device (102) receives training datasets from a health records server (108A), an insurance claims server (108B), and other third party servers (108C). DP computing device builds a model based on the training datasets and stores the model on a database (106) via a database server (104). Using the model and a stochastic learning algorithm, a risk estimator (110) determines a prediction of a disease or disorder diagnosis of a patient to a client device (112). The prediction is based on data gathered pertaining to the patient including unprocessed raw data comprising records of diagnostic codes generated during past medical encounters from an insurance claims database.

### CROSS-REFERENCE TO RELATED APPLICATIONS

This application claims priority to U.S. Provisional Application No. 62/937,604, filed Nov. 19, 2019, and U.S. Provisional Application No. 62/904,220, filed Sep. 23, 2019, which are hereby incorporated by reference in their entireties.

### STATEMENT REGARDING FEDERALLY SPONSORED RESEARCH OR DEVELOPMENT

This invention was made with government support under grant no. HR0011-18-9-0043 awarded by The Department of Defense. The government has certain rights in the invention.

### FIELD

The present disclosure generally relates to the diagnosis of disorders, and, more specifically, to systems and methods of creating zero-burden digital biomarkers for a myriad of disorders and exploiting co-morbidity patterns to drive early intervention.

### BACKGROUND

Autism spectrum disorders (ASD) are a heterogeneous group of early-onset neurodevelopmental impairments characterized by deficits in language and communication, difficulties in social interactions, and occurrence of restricted, stereotypic and repetitive patterns of behavior or interests. The prevalence of ASD has risen dramatically in the United States from one in 10,000 in 1972 to one in 59 children in 2014, with boys diagnosed at nearly four times the rate of girls. Increased awareness and better diagnostic practices do not fully explain this trend. With possibly over 1% of individuals affected worldwide, ASD presents a serious social problem with an increasing global burden. While the neurobiological basis of autism remains poorly understood, a detailed assessment conducted by the US Centers for Disease Control and Prevention (CDC) demonstrated that autistic children experience much higher than expected rates of many diseases, including conditions related to dysregulation of immune pathways such as eczema, allergies, asthma; as well as ear and respiratory infections, gastrointestinal problems, developmental issues, severe headaches, migraines, and seizures.

Despite the dramatic rise in prevalence in the US and around the world, the etiology of autism is still unclear, with no confirmed laboratory tests, and no cure on the horizon. Current incomplete understanding of ASD pathogenesis, and the lack of reliable biomarkers often hampers early intervention, with serious negative impact on the future lives of the affected children. Since early intervention is demonstrably crucial for improved quality of life, and for avoiding serious life-threatening complications, early detection and diagnosis is of paramount importance.

Gestational diabetes (GDM) affects greater than seven percent of pregnancies in the United States and over 16% of all pregnancies worldwide. GDM is typically diagnosed during the first prenatal visit or at the 24-28 week mark via a complicated sequence of glucose challenge tests (GCT) and repeated blood-work, contributing to increased costs and patient/provider burden. GDM is hyperglycemia and is associated with substantially increased maternal risk for subsequent type 2 diabetes (T2D), along with adverse neonatal outcomes that include macrosomia, respiratory disorders, and metabolic dysbiosis. GDM also has long-term consequences for both the mother and the offspring, and has been linked to elevated risk of future obesity, impaired glucose metabolism, cardiovascular disease, and metabolic syndrome.

A lack of current consensus on the precise diagnostic criteria leads to a complicated sequence of GCT and potentially repeated blood-work, contributing to increased costs and patient/provider burden. Early diagnosis is crucially important in the light of the effectiveness of available interventions and lifestyle changes in improving the odds of avoiding GDM and/or reduce or eliminate insulin use, along with reduced maternal as well as newborn weight gain. The number of GDM cases of all pregnancies worldwide is rising; and therefore GDM poses a serious and costly health problem.

While the pathobiology of T2D is still unresolved, it is clear that T2D and GDM are manifestations of impaired insulin secretory mechanisms and the associated metabolic pathways, with substantial heterogeneity in risk factors and comorbidities. While the causal chain linking some of these factors are well-understood, others have less robust evidential backing. Computational approaches are now being used to successfully design risk assessment tools for complex clinical decision problems that fuse information from electronic health records (EHR) via machine learning (ML) algorithms. For example, researchers used ML to leverage a diverse set of curated features derived from comprehensive EHR data in a large patient cohort to predict GDM. The researchers achieved an area under the receiver operating characteristic curve (AUC) of 85% at pregnancy initiation (defined as 32 weeks before birth), and used greater than 2000 different features including records of previous pregnancies, geographical and ethnic backgrounds, familial diabetic history, glucose levels recorded in the past, laboratory test results from past pregnancies, and results from the GCT. However, even with a substantially improved prediction (baseline: 67% AUC with traditional risk factors and 74% with genetic biomarkers), the researchers, with a sensitivity of about 30% at 95% specificity, were limited as a standalone diagnostic tool. The need to have access to specific blood-work and laboratory test results to derive the necessary features raises the data-requirement burden for the tool, precluding applicability to patients who might lack such detailed information.

### BRIEF SUMMARY

The present embodiments may relate to, inter alia, systems and methods for estimating risk of a diagnosis of certain disorders, such as Autism Spectrum Disorders (ASD). The disclosed embodiments are not limited to the diagnosis of ASD. For example, other disorders may be detected as well through the estimation of a risk of diagnosis, such as Asperger's syndrome, Attention-deficit/hyperactivity (ADHD), Bipolar disorder, Post Traumatic Stress Disorder (PTSD), preeclampsia, and anorexia. In one embodiment, a computer-based method is provided for receiving one or more training patient datasets. Further, the method may provide for partitioning a human disease spectrum into categories. Additionally, the method may provide for generating categorical time series from the one or more training patient datasets. Additionally, the method may provide for constructing of statistical models, such as a set of Hidden Markov Models (HMMs) representing the categories, genders, a treatment cohort, and a control cohort based on the first training patient dataset. A further enhancement of the method may include computing a sequence likelihood defect (SLD) for each category and for each patient in the second training patient dataset based on the HMMs. The method may further include training a tree-based classifier based on features extracted from another one of the one or more training patient datasets, including at least the SLDs, to weight the features and constructing an estimator based on the HMMs and the weighted features. Additionally, the method may further include validating the estimator based on yet another of the one or more training patient datasets. The computer-based method may include additional, less, or alternate functionality, including that discussed elsewhere herein.

Additionally, or alternatively, the present embodiments may relate to, inter alia, systems and methods for predicting a diagnosis of gestational diabetes (GDM), Postpartum Diabetes, Preeclampsia, Anorexia, Alzheimer's, Manic Switch, Pulmonary Fibrosis, Parkinson's, Sudden Unexplained Death Syndrome in Epilepsy, or Head and Neck Cancer. In some embodiments, GDM predictions, for example, may be generated based on a stochastic learning algorithm using unprocessed raw data. The unprocessed raw data may include data extracted from records of diagnostic codes generated during past medical encounters, such as from a national US insurance claims database, or the like.

In at least one aspect, a method for estimating risk of a disease diagnosis by a computing device is disclosed. The method may include retrieving unprocessed raw data associated with a plurality of patients; building a model relating elements of the unprocessed raw data; storing the model in a memory device communicatively-coupled to the computing device; receiving patient-specific data associated with at least one patient of the plurality of patients; and predicting a likelihood of a disease diagnosis or a disorder diagnosis for the at least one patient of the plurality of patients using the model and a stochastic learning algorithm based upon the received patient-specific data. The computing device may include additional, less, or alternate functionality, including that discussed elsewhere herein.

In another aspect, a method of estimating risk of a diagnosis of autism spectrum disorders is by a disease prediction (DP) computing device disclosed. The method includes receiving a first training patient dataset, a second training patient dataset, and a third training patient dataset; partitioning a human disease spectrum into categories; generating categorical time series from the first training patient dataset; constructing a set of statistical models representing the categories, genders, a treatment cohort, and a control cohort based on the first training patient dataset; computing a sequence likelihood defect (SLD) for each category and for each patient in the second training patient dataset based on the statistical model; training a tree-based classifier based on features extracted from the second training patient dataset, including at least the SLDs, to weight the features; constructing an estimator based on the statistical model and the weighted features; and validating the estimator based on the third training patient dataset. The DP computing device may include additional, less, or alternate functionality, including that discussed elsewhere herein.

Advantages will become more apparent to those skilled in the art from the following description of the preferred embodiments that have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The figures described below depict various aspects of the systems and methods disclosed therein. It should be understood that each figure depicts an embodiment of a particular aspect of the disclosed systems and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements that are presently discussed, it being understood, however, that the present embodiments are not limited to the precise arrangements and are instrumentalities shown, wherein:

FIG. 1 illustrates an exemplary Diagnosis Prediction (DP) computing system in accordance with an exemplary embodiment of the present disclosure;

FIG. 2 illustrates an exemplary client computing device that may be used with the DP computing system illustrated in FIG. 1;

FIG. 3 illustrates an exemplary server computing system that may be used with the DP system illustrated in FIG. 1;

FIG. 4A illustrates an example flowchart showing an example computer-based method for training the DP computing system illustrated in FIG. 1;

FIG. 4B illustrates an example flowchart showing an example computer-based method for predicting a diagnosis using the DP computing system illustrated in FIG. 1;

FIGS. 5A-5E illustrate example Autism Spectrum Diagnosis occurrence patterns in accordance with one or more embodiments of the present disclosure;

FIGS. 6A-6E illustrate example predictive performance in accordance with one or more embodiments of the present disclosure;

FIGS. 7A-7F illustrate example variation of inferred risk in accordance with one or more embodiments of the present disclosure;

FIGS. 8A-8C illustrate example co-morbidity patterns in accordance with one or more embodiments of the present disclosure;

FIGS. 9A-9D illustrate example details of co-morbidity patterns in accordance with one or more embodiments of the present disclosure;

FIG. 10 illustrates example receiver operating characteristic (AUC) sensitivity ratings for additional disorders that may be predicted in accordance with one or more embodiments of the present disclosure;

FIGS. 11A-11D illustrate example prediction performance in accordance with one or more embodiments of the present disclosure;

FIGS. 12A-12F illustrate example predictive performance, risk variation over time, and model complexity in accordance with one or more embodiments of the present disclosure; and

FIGS. 13A-13C illustrate example co-morbidity spectra in accordance with one or more embodiments of the present disclosure.

The figures depict preferred embodiments for purposes of illustration only. One skilled in the art will readily recognize from the following discussion that alternative embodiments of the systems and methods illustrated herein may be employed without departing from the principles of the invention described herein.

### DETAILED DESCRIPTION

Embodiments of the systems and methods described herein provide an accurate prediction of comorbid risks to drive early intervention. A stochastic learning algorithm may be utilized to predict disorders accurately and also provide cues to disorders which may be misdiagnosed as a different disorder. While the exemplary embodiments include the predicting of Autism Spectrum Disorder (ASD) or Gestational Diabetes (GDM), the described embodiments are in no way meant to be limiting. For example, additional disorders may be predicted using the disclosed methods, including, but not limited to, disorders such as Asperger's syndrome, Attention-deficit/hyperactivity disorder (ADHD), Bipolar disorder, Post Traumatic Stress Disorder (PTSD), Postpartum Diabetes, Preeclampsia, Anorexia, Alzheimer's, Manic Switch, Pulmonary Fibrosis, Parkinson's, Sudden Unexplained Death Syndrome in Epilepsy, Head and Neck Cancer.

### Autism Spectrum Disorder

Embodiments of the systems and methods described herein provide software implemented digital biomarkers for predicting a diagnosis in patients prior to a clinical decision. A predicted diagnosis is then utilized to drive early intervention. For example, software-implemented digital biomarkers may predict an Autism Spectrum Disorder (ASD) diagnosis in children before a clinical decision is made. The systems and methods described herein have demonstrably preempted clinical diagnosis by over two years on average, driving early intervention and translating to significant cost savings. Lacking a confirmed laboratory test for ASD, a predictive diagnostic has the potential for immediate transformative impact on patient care. Even though ASD may be diagnosed as early as the age of two, children typically remain undiagnosed until after their fourth birthday. The exemplary embodiments described herein describe the systems and methods predicting an autism diagnosis. However, the example of an autism diagnosis is not and should not be considered limiting, but is merely shown to provide a better understanding of the invention. Other types of disorders may be predicted using the software-implemented digital biomarkers. Other disorders related to ASD that may be predicted include, for example, Angelman Syndrome, Fragile X Syndrome, Landau-Kleffner Syndrome, Prader-Willi Syndrome, Tardive Dyskinesia, Williams Syndrome, or the like.

Embodiments of the systems and methods described herein map medical history of individual children under 2.5 years to the risk of a future autism diagnosis. They do so reliably enough that the results are of clinical significance. The systems and methods described herein may use individual diagnostic codes, already recorded during regular doctor's visits, to build a reliable risk estimation pipeline based on sophisticated stochastic learning algorithms, that demonstrably identifies high risk children at 2-3 years of age with a corresponding area under the receiver operating characteristic curve (AUC) exceeding 80% for either gender (83.3% for males, and 81.4% for females for age 2-2.5 years). As a result, ASD co-morbidities may be leveraged—at no additional burden—to predict elevated risk with clinically useful reliability at the earliest childhood years, where intervention is the most effective. The disclosed embodiments may be expected to significantly reduce the median diagnostic age for ASD, with an immediate transformative impact on patient care.

Autism spectrum disorders are a heterogeneous group of early-onset neurodevelopmental impairments characterized by deficits in language and communication, and difficulties in social interactions. The prevalence of ASD has risen dramatically in the United States from one in 10,000 in 1972 to one in 59 children in 2014, with boys diagnosed at nearly four times the rate of girls. With possibly over one percent of individuals affected worldwide, ASD presents a serious social problem with an increasing global burden.

A detailed assessment conducted by the US Centers for Disease Control and Prevention (CDC) demonstrated that autistic children experience much higher than expected rates of many diseases, including conditions related to dysregulation of immune pathways such as eczema, allergies, asthma, as well as ear and respiratory infections, gastrointestinal problems, developmental issues, severe headaches, migraines, and seizures.

Lacking a confirmed laboratory test for ASD, such a predictive diagnostic capability has the potential for immediate transformative impact on patient care: even though ASD may be diagnosed as early as the age of two, children remain undiagnosed until after their fourth birthday.

Despite dramatic rise in prevalence in the US and around the world, the etiology of autism is still unclear, with no confirmed laboratory tests, and no cure on the horizon. The current incomplete understanding of ASD pathogenesis, and the lack of reliable biomarkers often hampers early intervention, with serious negative impact on the future lives of the affected children. Since early intervention is demonstrably crucial for improved quality of life, and for avoiding serious life-threatening complications, early diagnosis is of paramount importance.

Embodiments of the systems and methods described herein track the risk of an eventual ASD diagnosis, based simply on the information gathered during regular doctor's visits, thus eliminating critical diagnostic delays at no additional burden, and thus radically improving intervention and treatment of the children with ASD. In some embodiments, the created biomarkers described herein contribute to the knowledge of the etiology of the ASD, thus pushing forward the research.

Certain known methods of predicting ASD diagnosis utilize analysis of blood work done on toddlers to ascertain their ASD status. In contrast, improved performance is achieved using systems and methods described herein and without the additional burden of new blood work.

Embodiments of the systems and methods may include the exploitation of co-morbidities (such as conditions related to dysregulation of immune pathways such as eczema, allergies, asthma, as well as ear and respiratory infections, gastrointestinal problems, developmental issues, severe headaches, migraines, and seizures, or the like) to estimate the risk of childhood neuropsychiatric disorders on the autism spectrum. In some embodiments, sequences of diagnostic codes from past doctor's visits may be used by a risk estimator to reliably predict a possible eventual clinical diagnosis for individual patients.

Embodiments of the systems and methods include training and out-of-sample cross-validation using independent datasets. In some embodiments, independent sources of clinical incidence data may be used to train a predictive pipeline. Further, extensive and rigorous cross-validation of the predictive pipeline may be performed using held-back data in at least one dataset of the independent datasets. Some embodiments may include the investigation of the impact of any unmodeled biases in a database.

Some embodiments of the disclosed DP computing system may include time-series modeling of diagnostic history. For example, individual diagnostic histories may have long-term memory, implying that the order, frequency, and co-morbid interactions between diseases are potentially important for assessing the future risk of target phenotypes. The disclosed system may include analyzing patient-specific diagnostic code sequences. The diagnostic code sequences may comprise of representing a medical history of each patient as a set of stochastic generators for individual data streams.

In some embodiments of the disclosed DP computing system, the system may include the partitioning of the human disease spectrum. For example, the system may include partitioning the human disease spectrum into non-overlapping categories that may remain fixed during and throughout an analysis. For example, each category may be defined by a set of diagnostic codes, such as from the International Classification of Diseases, Ninth Revision (ICD9), see Table I. Diagnostic histories may be transformed to report only these categories, thereby reducing the number of distinct codes that the aforementioned predictive pipeline may need to handle and improving statistical power. By improving statistical power, the disclosed system's trade-offs may include: 1) the loss of distinction between disorders in the same category, and 2) some inherent subjectivity in determining the constituent ICD9 codes that define each category.

Some embodiments of the disclosed DP computing system may include the processing of raw diagnostic histories to generate data streams that report only the categories instead of the exact codes. For example, each patient may have his or her past medical history represented as a sequence. In some embodiments, individual patient histories may be mapped to a three-alphabet categorical time series corresponding to a disease category. Each patient may then be represented by a mapped trinary series.

In further embodiments of the disclosed DP computing system, the system may include model inference and a sequenced likelihood defect. For example, the mapped series may be stratified by gender, disease category, and ASD diagnosis-status and may be considered to be independent realizations or sample paths from relatively invariant stochastic dynamical systems. These systems may, for example, be modeled as HMMs from observed variations in each subpopulation of patients. The different models may be compact representations of patterns emerging in the mapped time series. In some embodiments, the relative differences in the models may be exploited to reliably infer the cohort-type of a new patient from their individual sequence of past diagnostic codes. Example patient counts in de-identified data are shown in Table III.

Some embodiments of the disclosed DP computing system may include a risk estimation pipeline with semi-supervised and supervised learning modules. In some embodiments, a risk estimation pipeline may operate on patient specific information limited to the gender and available diagnostic history from birth, for example. The risk estimation pipeline may produce an estimate of the relative risk of a diagnosis, such as an ASD diagnosis at a specific age. In some embodiments, the estimate may include an associated confidence value.

### Gestational Diabetes

In some embodiments of the disclosed DP computing system a model, including a stochastic learning algorithm, may be used to provide an estimate for a Gestational Diabetes (GDM) diagnosis. The prediction may be performed using unprocessed raw data comprising of records of diagnostic codes generated during past medical encounters. In some embodiments, a trained pipeline may map individual medical histories to a raw indicator of risk. The ability to preempt GDM months before conception opens new intervention possibilities, including risk management through diet and exercise, for example. Additionally, or alternatively, delaying pregnancy by a few weeks may reduce GDM risk for certain patients.

In some embodiments, a GDM prediction may be generated using no laboratory test results, medications, demographic information, or even familial information. In at least one implementation, a sensitivity greater than 83% at 95% specificity was achieved with the corresponding area under the receiver operating characteristic curve (AUC) of 96.87% and a positive predictive value (PPV) greater than 53% at the first prenatal visit for low-risk patients (n=648,784). The AUCs when evaluated one, two, and four months before the first prenatal visit is respectively 92.75%, 91.82%, and 89.97% for the same cohort. A general cohort with potentially high risk patients includes (n=670,417) AUCs of 95.42% was achieved at the first prenatal visit, degrading to 89.24%, 88.06%, and 86.08% at the subsequent time points. For a high-risk cohort (n=104,946), AUCs of 94.83%, 89.31%, 87.51%, and 85.80%, respectively, were achieved. Accurate GDM risk assessment months before pregnancy opens new intervention possibilities, including risk management through lifestyle changes, as well as delaying pregnancy by a few weeks to reduce GDM risk. High predictive performance may provide cues to serious disorders which are often misdiagnosed as GDM due to confounding symptology such as Cushing's disease and tumors of the adrenal gland.

In some embodiments, the DP computing system may utilize a commercial database, such as an insurance claims database. The database may include data from multiple insurance carriers, such as 150 insurance carriers and/or large self-insurance companies. An example data source may be the Truven Health Analytics MarketScan® Commercial Claims and Encounters Database. The database may include billions of claims records, such as up to 4.6 billion inpatient/outpatient service claims, if not more, where each service claim may include one or more diagnosis codes. The computing system may extract diagnostic histories of female patients to obtain a training dataset to build a model for use to make GDM predictions. Example target codes that may be used to identify GDM are shown in Table IV. Example extracted diagnostic histories of female patients subject to the exclusion criteria are shown in Table V.

In some embodiments, predicting GDM by the DP computing device may be a binary classification problem, wherein sequences of diagnostic codes are to be classified into positive and control categories. “Positive” may refer to women eventually diagnosed with GDM while being pregnant, as indicated by the presence of a clinical diagnosis (one of the target codes from Table IV) in their medical records within 32 weeks after the code for pregnancy appears. The control cohort may comprise of patients who do not develop GDM. In at least one example, the predictor may be trained using 4.4 M diagnostic records from n=640,417 patients with 10,991 codes. See Table VI for example cohort sizes. Additionally, or alternatively, codes may not be pre-selected or rejected based on their known or suspected comorbidity with diabetes.

In some embodiments, in addition to the positive and control cohorts, two non-exclusive sub-cohorts to demonstrate robustness. For example, a priori low-risk cohort and an endocrinological high risk sub-cohort may be included. The priori low-risk cohort may exclude patients with prior history of high risk diagnoses (including diabetes, obesity and other factors, see Table VI) from both the control as well as the positive categories. The endocrinological high risk sub-hort may include only patients with at least one medical encounter in the year leading up to pregnancy resulting in a endocrinological diagnosis for both the control and positive categories. In at least one example, the cohorts may be treated independently and predictive pipelines may be derived individually. The pipelines may have comparable performance. In some embodiments, 50% of patients may be randomly selected for training models in each case. The remaining patients may be held back for out-of-sample evaluation.

In some embodiments, off-the-shelf classifiers such as random forests, gradient boosting, and deep learning may be superseded by stochastic learning algorithms customized for pattern discovery in diagnostic sequences. In some embodiments, a disease spectrum may be partitioned into broad categories (e.g., 43 broad categories such as infectious diseases, immunologic disorders, endocrinal disorders, etc.). Each of the categories may comprise of a relatively large number of diagnostic codes aligning with the broad categories defined within the ICD framework. Additionally, or alternatively, some of the categories may consist of a single diagnostic code, such as {626} mapping to disorders of menstruation and abnormal bleeding, and some comprise small code sets indicative of related disorders (e.g., 655, 656, 646.8, 659), mapping to complications in previous pregnancies. Each patient, for example, may be represented by a number of distinct sparse time series, where each time series tracks an individual disease category (e.g., 43 time series for 43 broad categories). At the population level, disease-specific stochastic time series may be compressed into specialized Hidden Markov Models (HMM) known as Probabilistic Finite Automata separately for the control and the positive cohorts to identify distinctive patterns pertaining to elevated GDM risk. In some embodiments, an inference algorithm for these models does not presuppose a fixed structure, may b able to work with non-synchronized and variable length inputs, and may yield category-specific state spaces with connectivity and transition probabilities that reflect subtle differences in dynamical patterns of the diagnostic sequences in the control vs. the positive categories. Subtle deviations in patterns in stochastic sequences may be quantified as reflected by different models obtained in a PFSA inference step. For example, a generalization of KL divergence may be known as the likelihood defect.

In additional to category specific Markov models, a range of engineered features may be used. Engineered features may reflect various aspects of diagnostic histories and may include the proportion of weeks in which a diagnostic code may be generated, the maximum length of consecutive weeks with codes, and the maximum length of weeks with no codes. This may result in different features evaluated for each patient (e.g., 316 different features). Additionally, or alternatively, inferred patterns included as features may be used to train a second level predictor, such as a standard gradient boosting classifier, that learns to map individual patients to control or positive groups based on their similarity to the identified Markov models of category-specific diagnostic histories and other engineered features. In some embodiments, 50% of training data may be used for PFSA inference and the remaining 50% may be used for training a second level classifier.

In some embodiments, a trained pipeline may map individual medical histories to a raw indicator of risk. Predictions may be made against a determined decision threshold. A decision threshold may be determined by maximizing an F₁ score. The score may be the harmonic mean of sensitivity and specificity. Additionally, or alternatively, a balanced trade-off between Type 1 and Type 2 errors may be made. A relative risk may be a ratio of the raw risk to the decision threshold, and a value greater than 1 predicts a future GDM diagnosis.

In some embodiments, the two step learning algorithm set forth herein does not demand results from specific tests, or look for specific demographic, bio-molecular, physiological, and other parameters. The algorithm set forth relies on diagnostic history of patients including, but not limited to, unstructured sequences of labels pertaining to ICD codes, which are prone to noise, coding errors, and sparsity. Performance may be measured using standard metrics including, but not limited to, AUC, sensitivity, specificity, and PPV. Different cohorts may be evaluated for predictions made at different time-points, namely at the first prenatal visit, and one, two, and four months before pregnancy initiation, for example.

In some embodiments, additional population measures with potentially important clinical relevant may be computed. For example, the change in GDM risk with each new endocrine event in the months leading to pregnancy which might be used to offer individual recommendations to women thinking of pregnancy in the near future. Additionally, or alternatively, comorbidity spectra for GDM may be computed which illustrates statistically significant log-odds ratio of being in the true positive vs. the true negative sets upon being assigned specific diagnostic codes.

### Diagnosis Prediction Computing System

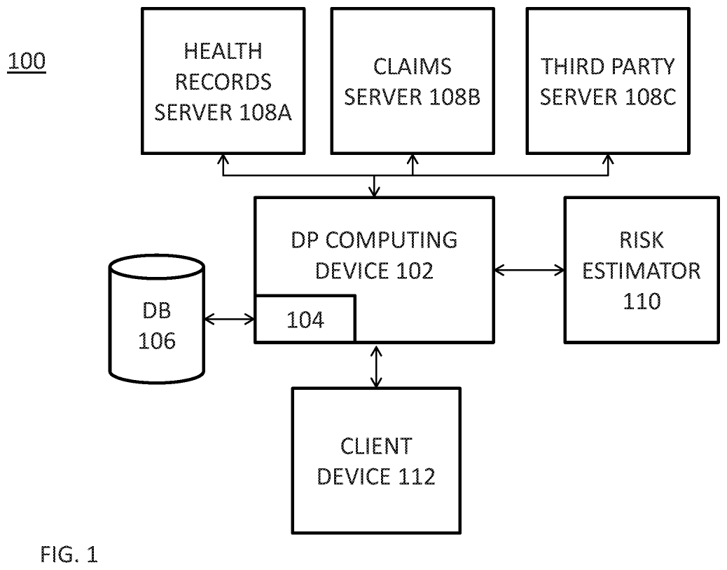

FIG. 1 depicts an example Diagnosis Prediction (DP) computing system 100. DP computing system 100 may include a DP computing device 102 (also referred to herein as DP server or DP computer device). DP computing device 102 may include a database server 104. DP computing device 102 may be in communication with, for example, one or more of a database 106, a Health Records server 108A, a claims server 108B, a third party server 108C, a Risk Estimator server 110, and a client computing device 112. In some embodiments, client device 112 may be a mobile computing device, a desktop computer, a laptop computer, a tablet PC, or the like. In some embodiments, DP computing device 102 may communicate with additional computing servers or client computing devices substantially similar to Health Records server 108, Risk Estimator server 110, and client device 112.

In an example embodiment, client device 112 may be a computer that includes a web browser or a software application, which enables the client device 112 to access remote computer devices, such as DP computing device 102, using the Internet or other network. More specifically, client device 112 may be communicatively coupled to DP computing device 102 through many interfaces including, but not limited to, at least one of the Internet, a network, such as the Internet, a local area network (LAN), a wide area network (WAN), or an integrated services digital network (ISDN), a dial-up-connection, a digital subscriber line (DSL), a cellular phone connection, and a cable modem. Client device 112 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices. In the exemplary embodiment, client device 208 may be associated with a user of the system, and the user may be a patient or associated with a patient undergoing DP prediction, such as a parent/guardian of a patient. While a single client computing device is shown, it is understood that more than one client device may be used in conjunction with the system. For simplicity, a single client device is shown merely as an example and is not meant to be limiting.

Database server 104 may be communicatively coupled to database 206 that stores data. In one embodiment, database 106 may include data received from Health Records Server 108A, claims server 108B, third party server 108C, Risk Estimator server 110, and/or client computing device 112.

In some embodiments, Health Records server 108A may be a single server or a plurality of different health records servers, such as electronic health records (EHRs) servers. Example EHRs may include the Truven Health Analytics MarketScan®, the Clinical Research Data Warehouse (CDRW), or even patient health records data as needed to perform DP prediction methods set forth herein.

In some embodiments, claims server 108B may be a single server or a plurality of different insurance claim servers that store historical insurance claims data. Example insurance claim servers may access claims databases such as a national insurance claims database. An insurance claims database may include, but is not limited to, raw data comprising records of diagnostic codes. Additionally, or alternatively, the diagnostic codes may be generated during past medical encounters.

In some embodiments, a third party server 108C may be accessed by the DP computing device 102 to access other data that may be provided beyond what is accessible from health records server 108A and claims server 108B. Other data may include historical or archived medical records, prior training datasets, or the like.

In some embodiments, Risk Estimator 110 may be trained and models created based on a plurality of datasets gathered from servers 108A, 108B, and/or 108C described herein. Further, cross-validation may be performed by the Estimator 110 to validate one or more of the datasets. Risk Estimator 110 may then calculate a patient's likelihood of a disorder, such as ASD or GDM described above based on a pipeline of data in view of the models created, along with other data pertaining to or relevant to the patient. The calculation of risk may include a stochastic learning algorithm for predicting a patient's likelihood of a disorder.

### Exemplary Client Computing Device

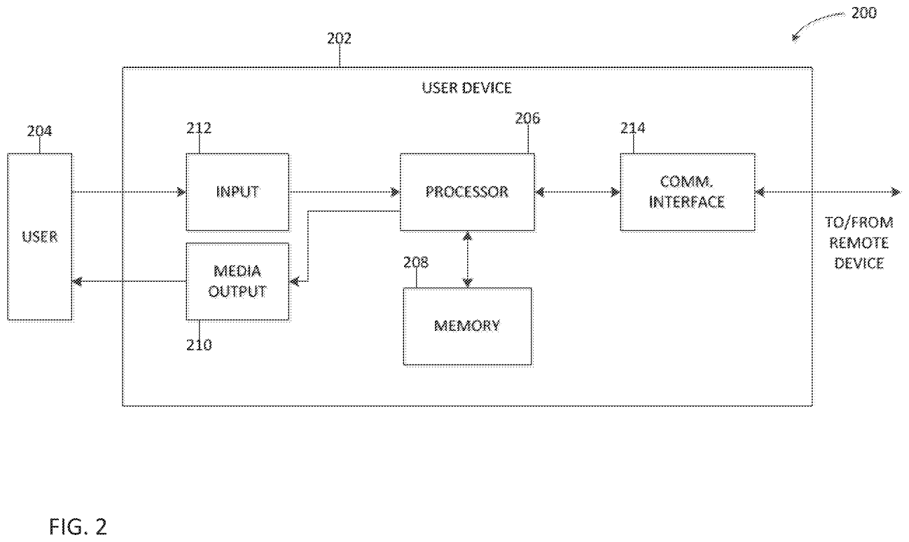

FIG. 2 depicts an exemplary diagram 200 illustrating client computing device 202 that may be used with DP computing system 100 shown in FIG. 1. Client computing device 202 may be, for example, at least one of DP computing device 102, client device 112, and/or risk estimator device 110 (all shown in FIG. 1).

Client computing device 202 may include a processor 205 for executing instructions. In some embodiments, executable instructions may be stored in a memory area 208. Processor 206 may include one or more processing units (e.g., in a multi-core configuration). Memory area 208 may be any device allowing information such as executable instructions and/or other data to be stored and retrieved. Memory area 208 may include one or more computer readable media.

In exemplary embodiments, client computing device 202 may also include at least one media output component 210 for presenting information to a user 204. Media output component 210 may be any component capable of conveying information to user 204. In some embodiments, media output component 210 may include an output adapter such as a video adapter and/or an audio adapter. An output adapter may be operatively coupled to processor 206 and operatively couplable to an output device such as a display device (e.g., a liquid crystal display (LCD), light emitting diode (LED) display, organic light emitting diode (OLED) display, cathode ray tube (CRT) display, “electronic ink” display, or a projected display) or an audio output device (e.g., a speaker or headphones). Media output component 210 may be configured to, for example, display an alert message identifying a statement as potentially false.

Client computing device 202 may also include an input device 212 for receiving input from user 204. Input device 212 may include, for example, a keyboard, a pointing device, a mouse, a stylus, a touch sensitive panel (e.g., a touch pad or a touch screen), a position detector, or an audio input device. A single component such as a touch screen may function as both an output device of media output component 210 and input device 212.

Client computing device 202 may also include a communication interface 214, which can be communicatively coupled to a remote device such as DP computing device 102 (shown in FIG. 1). Communication interface 214 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 208 may be, for example, computer-readable instructions for providing a user interface to user 204 via media output component 210 and, optionally, receiving and processing input from input device 212. A user interface may include, among other possibilities, a web browser and client application. Web browsers may enable users, such as user 204, to display and interact with media and other information typically embedded on a web page or a website.

Memory area 208 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

### Exemplary Server Computing Device

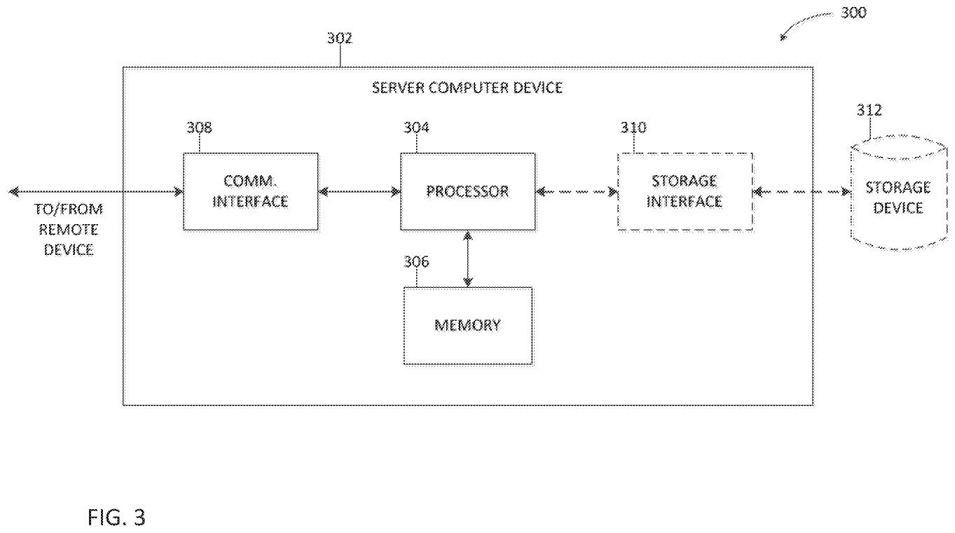

FIG. 3 depicts diagram 300 illustrating exemplary server system 302 that may be used with DP computing system 100 illustrated in FIG. 1. Server system 302 may be, for example, server computing device 108A, 108B, or 108C (shown in FIG. 1).

In exemplary embodiments, server system 302 may include a processor 304 for executing instructions. Instructions may be stored in a memory area 306. Processor 304 may include one or more processing units (e.g., in a multi-core configuration) for executing instructions. The instructions may be executed within a variety of different operating systems on server system 302, such as UNIX, LINUX, Microsoft Windows®, etc. It should also be appreciated that upon initiation of a computer-based method, various instructions may be executed during initialization. Some operations may be required in order to perform one or more processes described herein, while other operations may be more general and/or specific to a particular programming language (e.g., C, C #, C++, Java, or other suitable programming languages, etc.).

Processor 304 may be operatively coupled to a communication interface 308 such that server system 302 is capable of communicating with DP computing device 102, client device 112, and/or the risk estimator device 110 (all shown in FIG. 1), and/or another server system 302. For example, communication interface 308 may receive requests from client device 112 via the Internet.

Processor 304 may also be operatively coupled to a storage device 312, such as database 106 (shown in FIG. 1). Storage device 312 may be any computer-operated hardware suitable for storing and/or retrieving data. In some embodiments, storage device 312 may be integrated in server system 302. For example, server system 302 may include one or more hard disk drives as storage device 312. In other embodiments, storage device 312 may be external to server system 302 and may be accessed by a plurality of server systems 302. For example, storage device 312 may include multiple storage units such as hard disks or solid state disks in a redundant array of inexpensive disks (RAID) configuration. Storage device 312 may include a storage area network (SAN) and/or a network attached storage (NAS) system.

In some embodiments, processor 304 may be operatively coupled to storage device 312 via a storage interface 310. Storage interface 310 may be any component capable of providing processor 304 with access to storage device 312. Storage interface 310 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 305 with access to storage device 312.

Memory area 306 may include, but is not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are exemplary only, and are thus not limiting as to the types of memory usable for storage of a computer program.

### Exemplary Method for Building Model

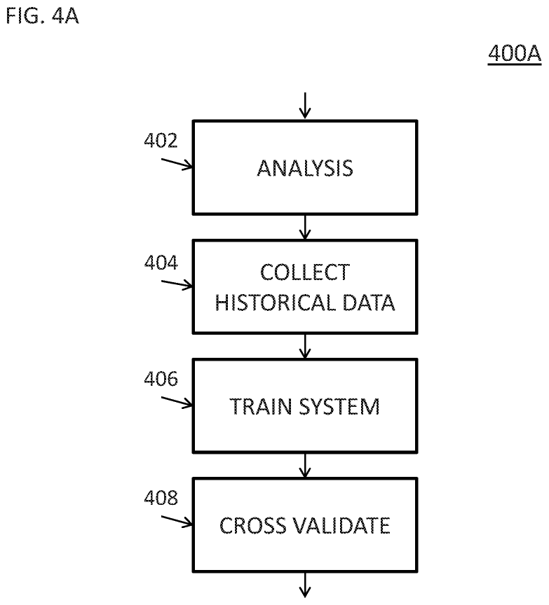

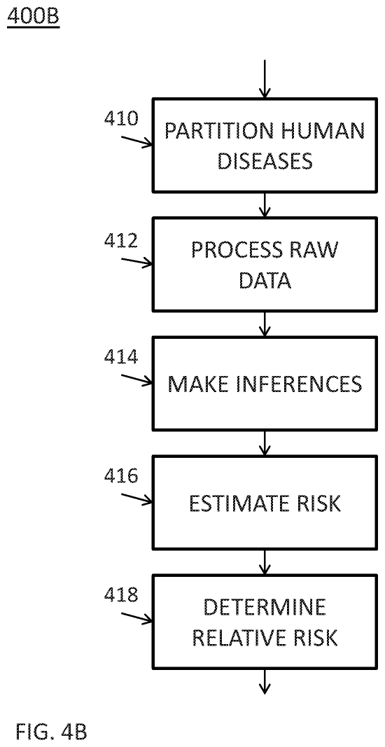

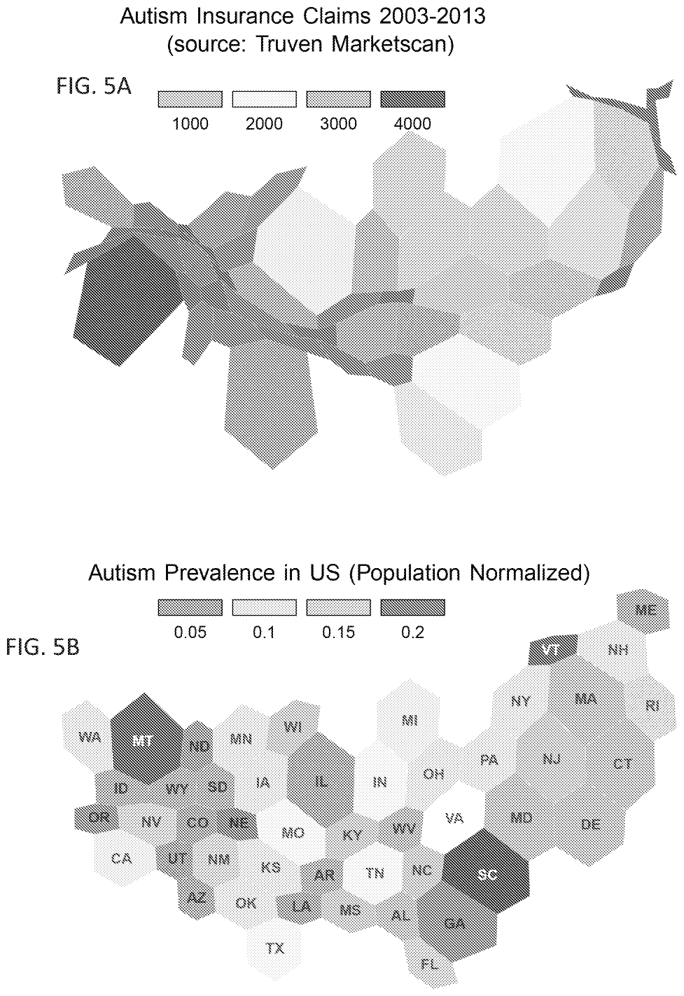

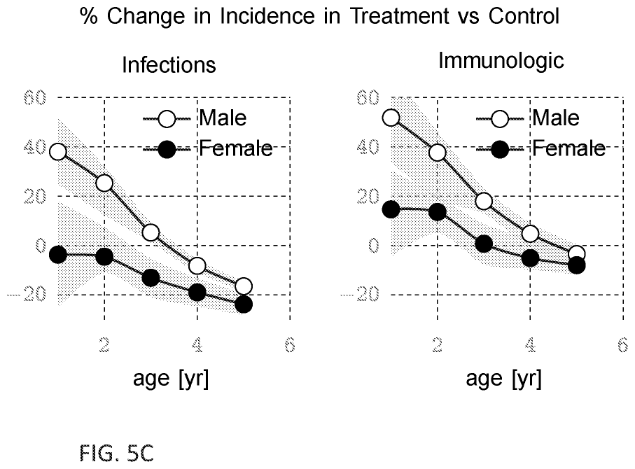

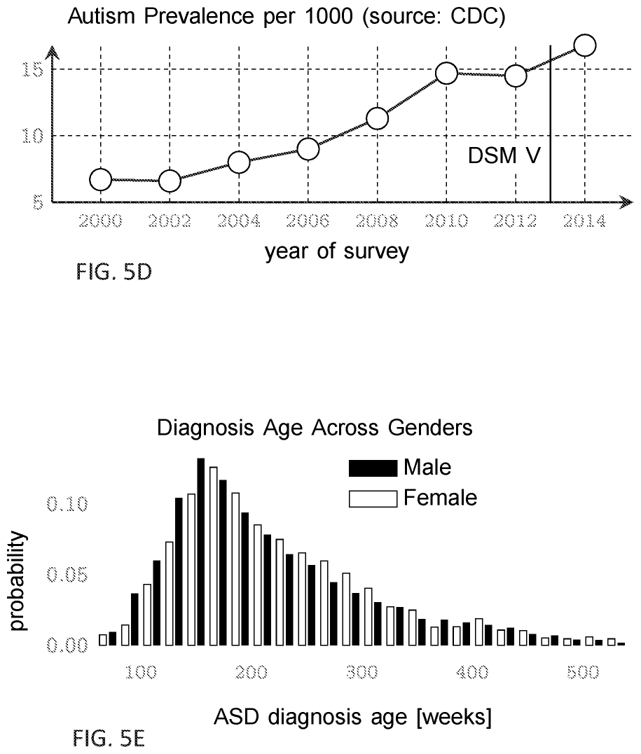

In reference to FIG. 4A, flow 400A, analysis 402 on two independent electronic databases of diagnostic histories may be used: 1) a claims database for private health insurance, such as Truven Marketscan, tracking over 5.6 million children between 2003 and 2012; and 2) a set of de-identified diagnostic records, such as diagnostic records for nearly 70 thousand children under 5 years of age treated at the University of Chicago Medical Center between 2006 and 2018. The claims database may be in good agreement with documented prevalence: consistent with independent reports that ASD prevalence is weakly dependent on demographic variables, and that such effects are progressively diminishing, it is found that the distortion in the cartogram in FIG. 5A, illustrates a spatial skew of prevalence in the dataset largely disappears after population normalization (FIG. 5B). Additionally, in agreement with the widely studied ASD co-morbidity burden, infections and immunological disorders have differential representation in the treatment and control groups (FIG. 5C). The median diagnosis age may also be comparable, around three years in the claims database (FIG. 5E) versus three years, ten months for ASD in the US.

Currently, over one hundred genes have been shown to contribute to autism risk, and it is estimated that up to 1,000 genes might be involved in ASD pathogenesis. Nevertheless, genetic interactions and mechanisms have accounted for a limited number of ASD cases, potentially implicating environmental triggers that work alongside genetic predispositions. Plausible sources of risk may range from prenatal factors such as maternal infection and inflammation, diet, household chemical exposures, to autoimmune conditions and localized inflammation of the central nervous system after birth. The heterogeneity of ASD presentation also admits the possibility of a plurality of etiologies with converging pathophysiological pathways, making the investigation of the etiology of future risk modulation extremely challenging. Furthermore, standard machine learning tools fail to achieve meaningful performance. Further, the available data is too sparse for off-the-shelf deep learning frameworks to make personalized predictions, and standalone classifiers or regressors fail to exploit the temporal dynamics embedded in the sparse diagnostic histories, requiring a new and improved machine inference algorithms and feature engineering approaches to distill effective risk predictors.

Flow 400A may include collecting 404 electronic patient records from independent sources of clinical incidence data. Collected clinical incidence data may be used for training 406 a predictive pipeline. An example source, which will be referred to as an example dataset to illustrate the disclosed, may be the Truven Health Analytics MarketScan® Commercial Claims and Encounters Database for the years 2003 to 2012 (“Truven dataset”). The Truven dataset comprises of approved commercial health insurance claims for between 17.5 and 45.2 million people annually, with linkage across years, for a total of approximately 150 million individuals. The Truven dataset contains data contributed by over 150 insurance carriers and large, self-insuring companies. The Truven dataset includes 4.6 billion inpatient and outpatient service claims and approximately six billion diagnosis codes. For the disclosed analysis, histories of patients within the age of 0-9 may be extracted and may exclude patients lacking with at least one diagnostic code without a set of specified disease categories before the first 30 weeks of life.

A dataset, such as the Truven dataset, may be used for both the training and out-of-sample cross-validation 408 with held-back patient data. A second independent dataset may aid in further cross-validation. A second set of patient data, such as a UCM dataset may be provided by the Clinical Research Data Warehouse (CDRW) maintained by the Center for Research Informatics (CRI) at University of Chicago. Post-construction, extensive and rigorous cross-validation of the predictive pipeline with held-back data in the Truven dataset. To investigate the impact of any unmodeled biases in the database, re-validation may be performed on the results on the UCM dataset. This example of number of patients used from the two datasets is shown in Table III.

Individual diagnostic histories may have long-term memory, implying that the order, frequency, and comorbid interactions between diseases may be potentially important for assessing the future risk of a target phenotype. At least one approach to analyzing patient-specific diagnostic code sequences consists of representing the medical history of each patient as a set of stochastic categorical time-series, one each for a specific group of related disorders, followed by an inference of stochastic generators for these individual data streams. These inferred generators may be from a special class of Hidden Markov Models (HMMs), referred to as Probabilistic Finite State Automata (PFSA). Next, the cross-validation may include the inference of a separate class of models for the treatment and control cohorts, and then the problem reduces to determining the probability that the short diagnostic history from a new patient arises from the treatment as opposed to the control category of the inferred models. Importantly, the individual patient histories may be typically short, often have large randomly varying gaps, and have no guarantee that model-structural assumptions (e.g., linearity, additive noise structure, etc.) often used in the standard time-series analysis is applicable. The categorical observations may be drawn from a large alphabet of possible diagnostic codes, which degrades statistical power. Patterns emergent at the population level to make individual risk assessments is challenged by the ecological fallacy, that group statistics might be neither reflective nor predictive of patterns at the individual level.

In accordance with the workflow diagram 400B of FIG. 4B, a first step in accordance with one or more embodiments of the disclosed invention may include partitioning of the human disease spectrum (step 410). For example, to address the idiosyncrasies of the problem to be solved, the first step is to partition the human disease spectrum into non-overlapping categories, such as 15 non-overlapping categories (see Table I), for example. The non-overlapping categories may remain fixed throughout the analysis. Each category may be defined by a set of diagnostic codes from the International Classification of Diseases, Ninth Revision (ICD9). Transforming the diagnostic histories to report only these categories may reduce the number of distinct codes that the pipeline needs to handle, thus improving statistical power. The trade-offs for this increased power consist of 1) the loss of distinction between disorders in the same category, and 2) some inherent subjectivity in determining the constituent ICD9 codes that define each category (e.g., an ear infection may be classified either as an otic disease or an infectious one). Exemplary disease categories, along with example ICD9 codes, are shown in Table I. Exemplary engineered features of the disease categories are shown in Table II.

Next, flow 400B may include processing 412 of raw data streams to generate data streams that report only the categories instead of the exact codes. For each patient, his or her past medical history may be a sequence (t₁, x₁), . . . , (tₘ, xₘ), where tᵢ are timestamps and xᵢ are ICD9 codes diagnosed at time tᵢ. Individual patient histories may be mapped to a three-alphabet categorical time series z^{k} corresponding to a disease category k, as follows. For each week i, we have:

$${z}_{i}^{k} = \{ \begin{matrix}0 & if no diagnosis codes in week i \\ 1 & if there exists a diagnosis of category k in week i \\ 2 & otherwise\end{matrix}$$

A time series z^{k} may be terminated at a particular week if the patient is diagnosed with ASD the week after. Thus for patients in the control cohort, the length of the mapped trinary series may be limited by the time for which the individual is observed within a certain time span, such as within 2003-2012 time span. In contrast, for example, patients in the treatment cohort, the length of the mapped series may reflect the time to a first ASD diagnosis. Typically, patients do not necessarily enter the database at birth. Each series may be prefixed with 0 s to approximately synchronize observations to age in weeks. An approximation may arise from the absence of exact birthdays in a certain database, wherein an uncertainty, such as 0.5 years, may exist for all time estimates.

Step 412 of flow 400B may then represent each patient by a mapped trinary series, such as 15 mapped trinary series for example, and used to infer 414 population-level PFSA models. For example, each mapped series may be stratified by gender, disease-category, and ASD diagnosis status and may be considered to be independent realizations or sample paths from relatively invariant stochastic dynamical systems. The dynamical systems may be modeled as statistical models, such as HMMs, from the observed variations in each subpopulation of patients. Model inference may include modeling of the treatment and the control cohorts for each gender, and in each disease category separately, for example, and ending up with a total of 60 HMMs at the population level, when there are 15 categories, two genders, two cohort-types (e.g., treatment and control). Each of the inferred models may be a PFSA including a directed graph with probability weighted edges, and may act as an optimal generator of the stochastic process driving the sequential appearance of the three letters corresponding to each gender, disease category, and cohort-type. The models may be very nearly assumption-free beyond a requirement that the processes be statistically stationary or slowly varying. The models may be not apriori constrained by any structural motifs, complexity, or size, and may be compact representations of patterns emerging in the mapped time series. Relative differences may be exploited in the probabilistic models to reliably infer the cohort type of a new patient from their individual sequence of past diagnostic codes.

The Kullbeck-Leibler (KL) divergence may be used. For example, the KL divergence between probability distributions to a divergence D_{KL}(G∥H) between ergodic stationary categorical stochastic processes G,H, as:

$${𝒟}_{KL} ( G ‖ H ) = {lim}_{n \to ∞} \frac{1}{n} {\sum }_{x \vert x \vert = n} {p}_{G} ( x ) log \frac{{p}_{G} ( x )}{{p}_{H} ( x )}$$

where |x| is the sequence length, and p_{G}(x),p_{H}(x) are the probabilities of sequence x being generated by the processes G, H respectively. Defining the log-likelihood of x being generated by a process G as:

$$L ( G , x ) = - \frac{1}{\vert x \vert} log {p}_{G ( x )}$$

the cohort-type for an observed sequence x—which may be actually generated by the hidden process G—can then formally be inferred from observations based on the following provable relationships:

$${lim}_{\vert x \vert \to ∞} L ( G , x ) = ℋ ( G )$$

$${lim}_{\vert x \vert \to ∞} L ( H , x ) = ℋ ( G ) + {𝒟}_{KL} ( G ‖ H )$$

where ′H is the entropy rate of a process. The above equation shows that the computed likelihood has an additional non-negative contribution from the divergence term, when the incorrect generative process is chosen. Thus, if a patient is eventually going to be diagnosed with ASD, then it may be expected that the disease-specific mapped series corresponding to his or her diagnostic history may be modeled by the PFSA in the treatment cohort. Denoting the PFSA corresponding to disease category j for treatment and control cohorts as G^{j}₊, G^{j}₀ respectively, the sequence likelihood defect (SLD, Δ^{j}) may be computed as:

Δ^{j}±L(G^{j}₀,x)−L(G₊₁^{j},x)→ [symbol] _{α}(G₀^{j}∥G₊^{j})

Based on the inferred population-level PFSA models and individual diagnostic history, the SLD measure can now be estimated. The higher this likelihood defect, the higher the similarity of a certain patient's history to others that have had an eventual ASD diagnosis, with respect to the disease category being considered. With respect to a risk estimation pipeline, the SLD may be considered to be a core analytic tool used to tease out information relevant to the risk estimator.

Flow 400B may continue with the producing 416 of a risk estimation of an ASD diagnosis. The risk estimation pipeline may include one or more semi-supervised and supervised learning modules. The risk estimation pipeline may operate on patient specific information limited to the gender and available diagnostic history from birth. The pipeline may produce an estimate of the relative risk of ASD diagnosis at a specific age along with an associated confidence value. The parameters and associated model structures of the pipeline may be transformed by the patient specific data to a set of engineered features, and the feature vectors realized on the treatment and control sets may be then used to train a gradient-boosting classifier. The set of engineered features may include the disease-category-specific SLD described above. For example, if SLD>0 for a specific patient for every disease category, then he or she is likely to have an ASD diagnosis eventually. However, not all disease categories are equally important for such a decision. For example, parametric tuning of the classifier may allow for the inference of optimal combination weights, as well as the computation of relative risk with associated confidence. In addition to category-specific SLDs, a range of other derived quantities as features may be used, including the mean and variance of the defects computed over all disease categories, the occurrence frequency of the different disease groups, etc. An example list of features that may be used by the estimation pipeline is provided in Table II.

In some embodiments, the HMM models may need to be inferred prior to the calculation of the likelihood defects. For example, two training sets may be used, one that is used to infer the models and one that subsequently trains the classifier in the pipeline with features derived from the inferred models. The analysis may proceed by first carrying out a random 3-way split of the set of unique patient data IDs into feature-engineering (25%), training (25%) and test (50%) sets. A feature-engineering set of ids may be used to first infer a number of PFSA models, such as unsupervised model inference in each category, which then may allow training of a gradient-boosting classifier using the training set and PFSA models, such as classical supervised learning, and finally carry out out-of-sample validation on the test set. Appropriate sizes of the three example sets may be as follows: ˜700K each for the feature-engineering and the training sets, and ˜1.5 M for the test set. The features used in the pipeline may be ranked in order of their relative importance (See FIG. 5, Plate E), by estimating the loss in performance when dropped out of the analysis.

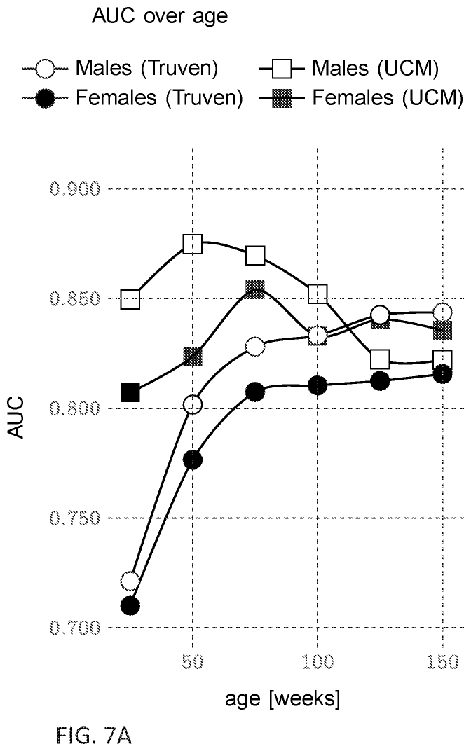

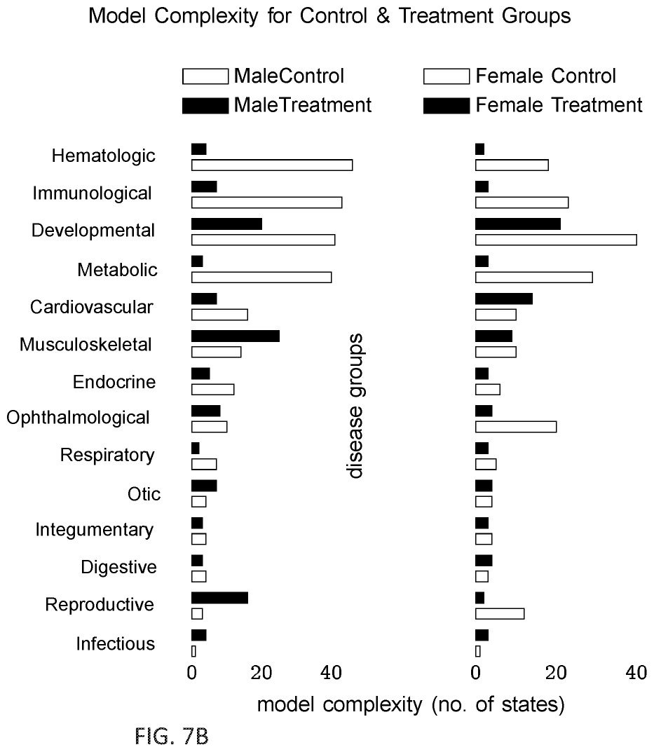

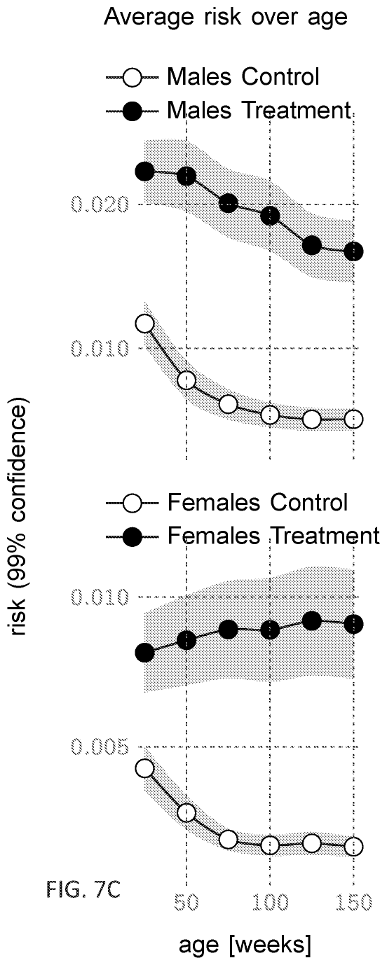

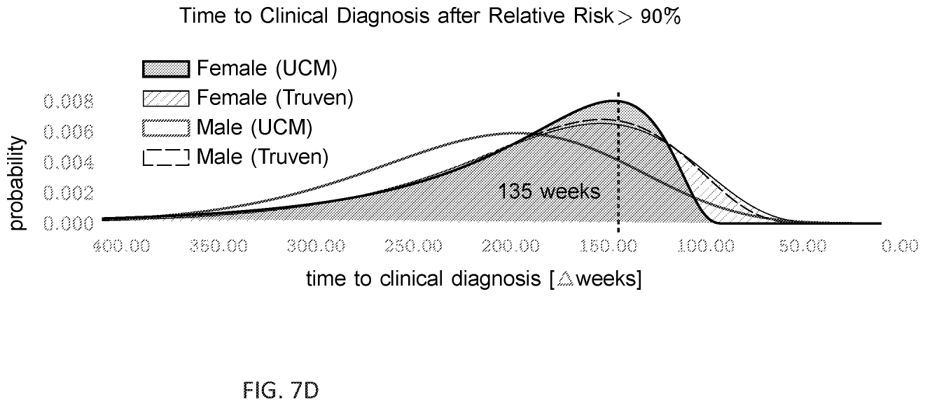

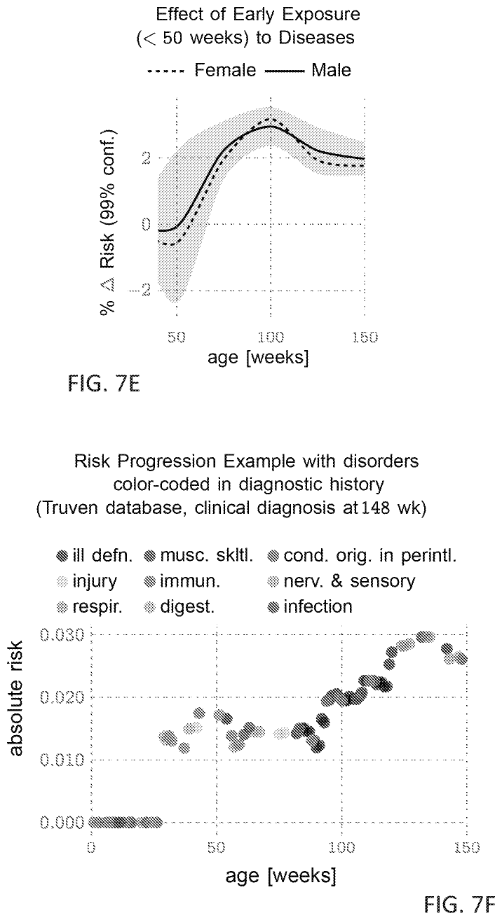

The DP computing device 102 may further determine 418 a relative risk by mapping medical histories to a score, which is interpreted as a raw indicator of risk. For example, the higher the score, the higher the probability of a future diagnosis. A decision threshold may be chosen for the raw score. For example, conceptually identical to the notion of Type 1 and Type 2 errors in classical statistical analyses, the choice of a threshold trades off false positives, a Type 1 error, for false negatives, a Type 2 error. The choosing of a small threshold results in predicting a larger fraction of future diagnoses correctly (i.e. have a high true positive rate (TPR)), while simultaneously suffering from a higher false positive rate (FPR), and vice versa. A receiver operating characteristic curve (ROC) may be the plot of the FPR vs. the TPR, and may vary the decision threshold. If the predictor is determined to be good, then it is determined that the system consistently achieves high TPR with small FPR resulting in a high area under the ROC curve (AUC). AUC may measure intrinsic performance, independent of the threshold choice. The AUC is typically immune to class imbalance in view of the fact that the control cohort is several orders of magnitude larger than the treatment cohort. For example, an AUC of 50% indicates that the predictor does no better than random, and an AUC of 100% implies that perfect prediction of future diagnoses is achieved with zero false positives. Example reported AUCs are shown in FIG. 7A. In this example, the reported AUCs were all computed on out-of-sample data (i.e. on held back subset for the Truven database, and on the entirety of the UCM samples, the latter being never used in training and pipeline design).

The choice of a certain decision threshold is considered necessary for making individual predictions and meaningful risk assessments thereby reflecting a choice of the maximum FPR and minimum TPR. An analysis may be based on maximizing the F₁-score, defined as the harmonic mean of sensitivity and specificity, to make a balanced tradeoff between the two kinds of errors. Other strategies for selecting thresholds may include maximizing accuracy, the fraction of correct predictions on the presence of absence of future diagnosis, or maximizing the true positives rate or the recall of the decision maker.

In accordance with one or more embodiments, relative risk may be defined as the ratio of a raw pipeline score to a chosen decision threshold. Thus, a relative risk >1 implies the prediction of an eventual ASD diagnosis, and on average, decisions maximize the F₁-score of the pipeline. A raw score typically does not, by itself, give actionable information, the relative risk being close to or greater than 1.0 for a specific patient signals the need for intervention.

FIGS. 5A-5E illustrate ASD occurrence patterns in accordance with one or more embodiments of the disclosure. As shown, FIG. 5A illustrates the spatial distribution of ASD insurance claims; FIG. 5B illustrates the same data after population normalization, illustrating the relatively small demographic skew to ASD prevalence; FIG. 5C illustrates the recent dramatic increase in prevalence as reported by the CDC; FIG. 5D illustrates the differential representation of different disease categories in the treatment and control cohorts; and FIG. 5E illustrates the distribution of the age of diagnosis for males and females. Further, FIG. 5E illustrates the sparsity of the available codes for individual subjects.

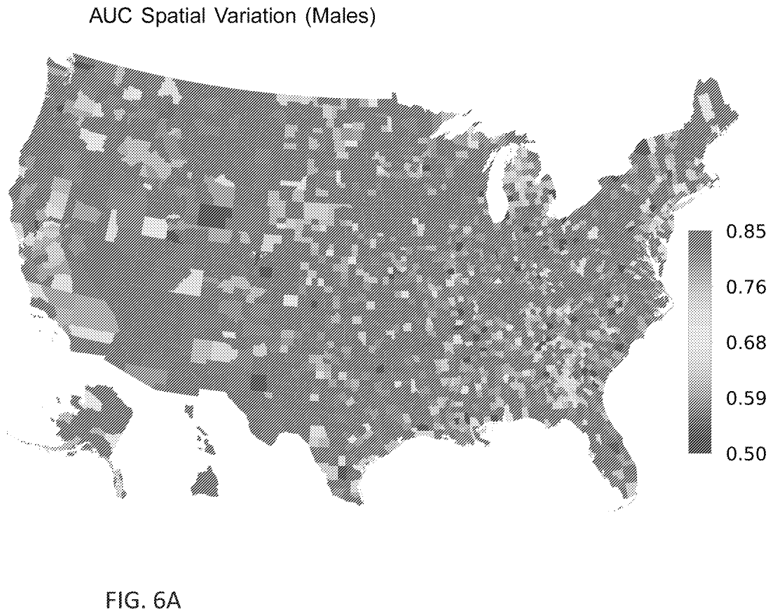

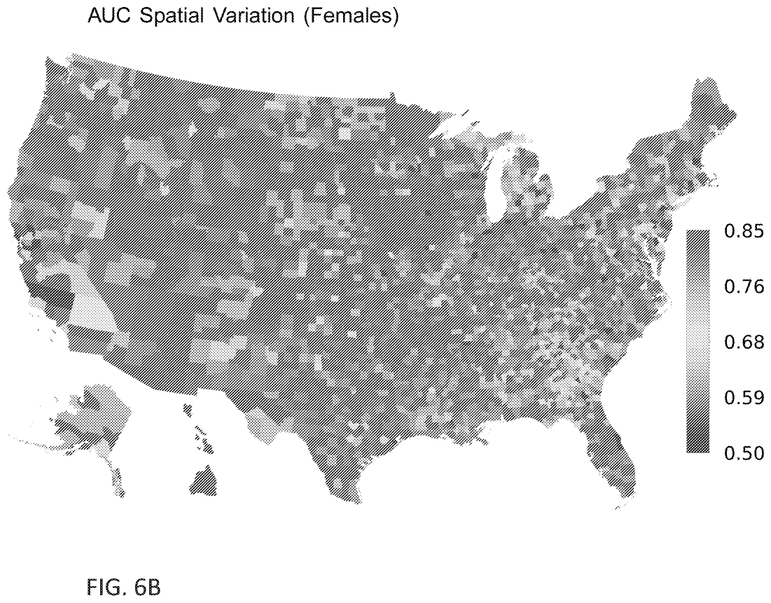

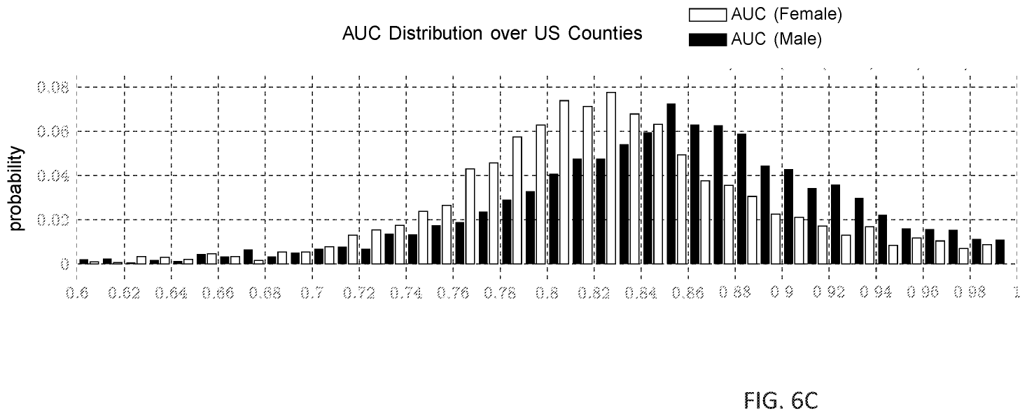

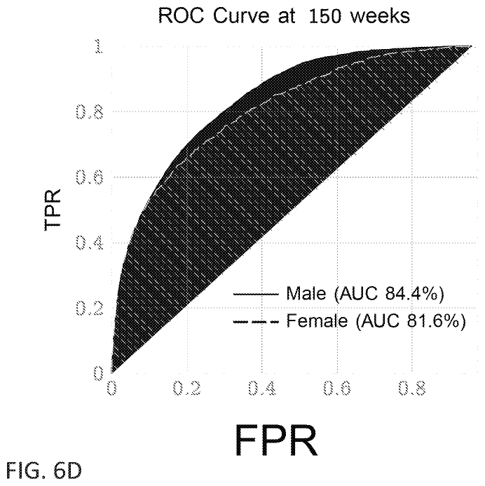

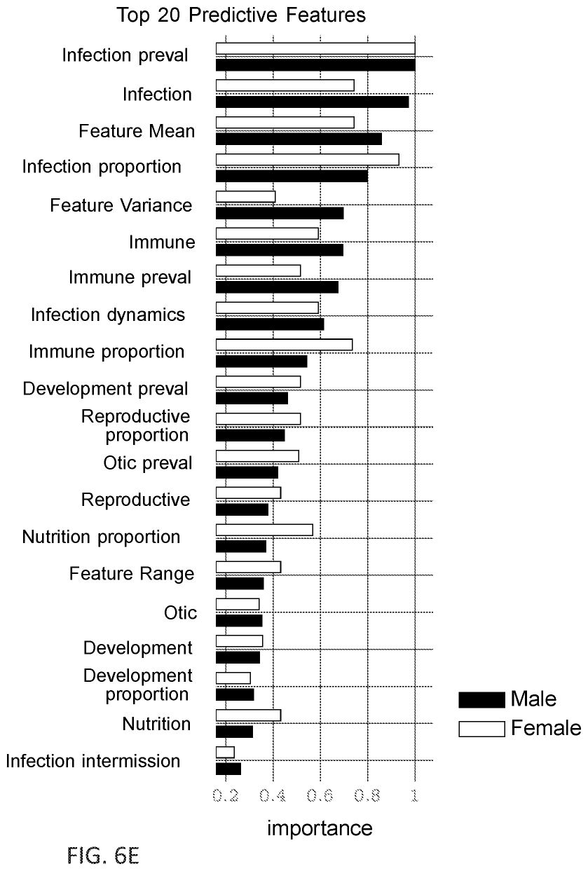

FIGS. 6A-6E illustrate predictive performance. FIGS. 6A and 6B show the spatial variation in the achieved predictive performance at 150 weeks, measured by AUC, for males and females, respectively. Gray areas lack data on either positive or negative cases. FIG. 6C shows the distribution of the AUC, and FIG. 6D shows the ROC curves for males and females. FIG. 6E shows the feature importance inferred by a prediction pipeline. The detailed description of the features is given in Table I. The most important feature for prediction is feature mean and feature variance, which are the mean and variance of a novel stochastic automata based metric, averaged over 18 phenotypically and etiologically distinct disease categories.

FIGS. 7A-F illustrate variation of inferred risk. FIG. 7A illustrates AUC achieved as a function of patient age, for the Truven and UCM datasets. The shaded area outlines the 2-2.5 years of age within which AUC>80% is achieved for either gender. FIG. 7B illustrates how inferred models differ between the control vs. the treatment cohorts. On average models get less complex, implying the exposures get more statistically independent. However, the models for some disease groups get more complex, indicating a stronger historical dependence. FIG. 7C illustrates how the average risk changes with time for the control and the positive cohorts. FIG. 7D shows the distribution of the prediction horizon: the time to a clinical diagnosis after inferred relative risk crosses 90%. FIG. 7E shows that for each new disease code for a low risk child. ASD risk increases by approximately 2% for either gender. FIG. 7F illustrates the risk progression of a specific, ultimately autistic male child in the Truven database.

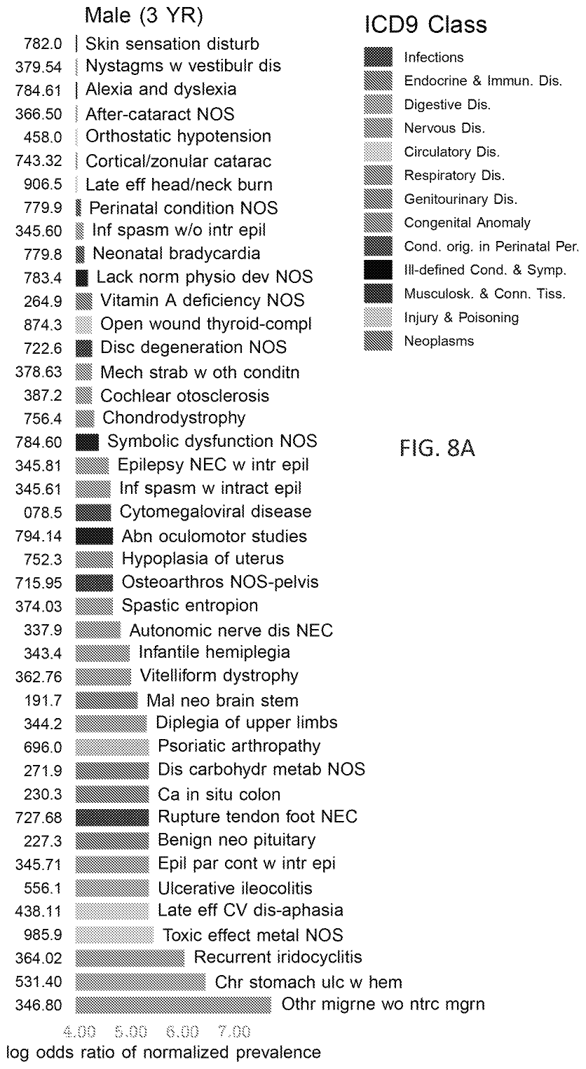

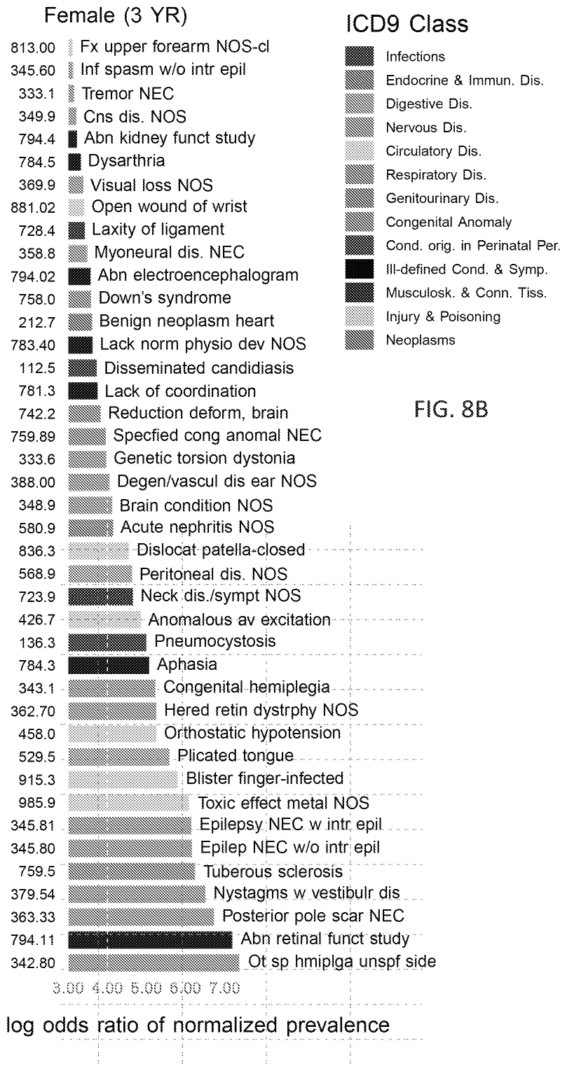

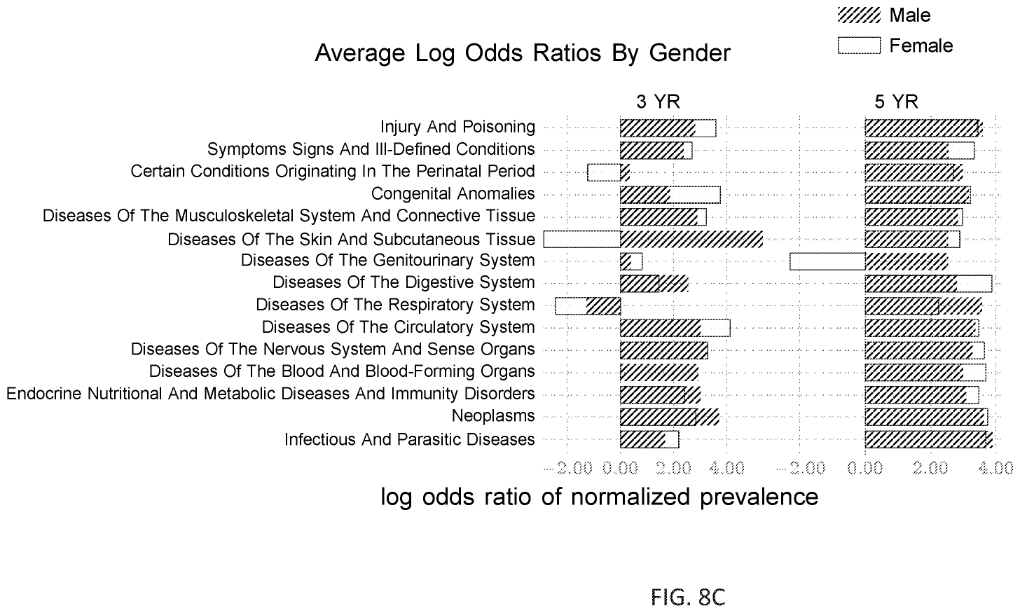

FIGS. 8A-8C illustrate co-morbidity patterns in FIGS. 8A and 8B. Further, a difference in occurrence frequencies of diagnostic codes between true positive (TP) and True Negative (TN) predictions is shown. The dotted line on FIG. 8B shows the abscissa lower cut-off in FIG. 8A, illustrating the lower prevalence of codes in females. FIG. 8C also illustrates log-odds ratios for ICD9 disease categories.

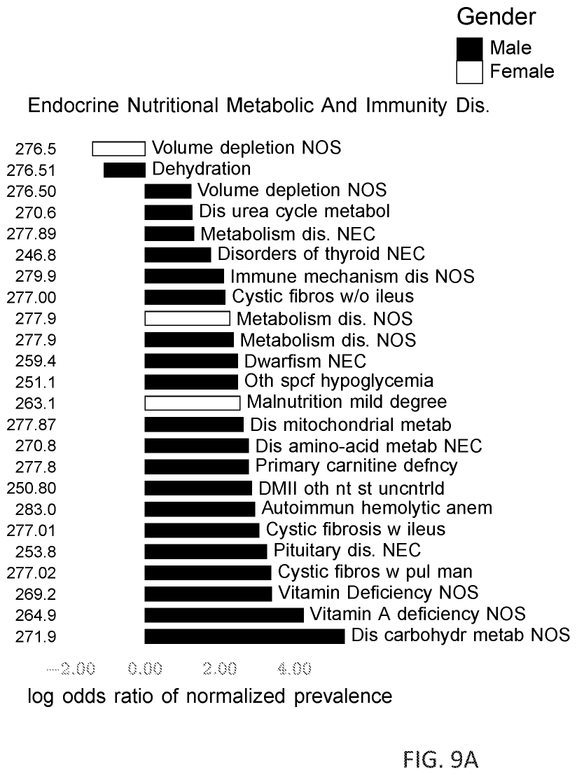

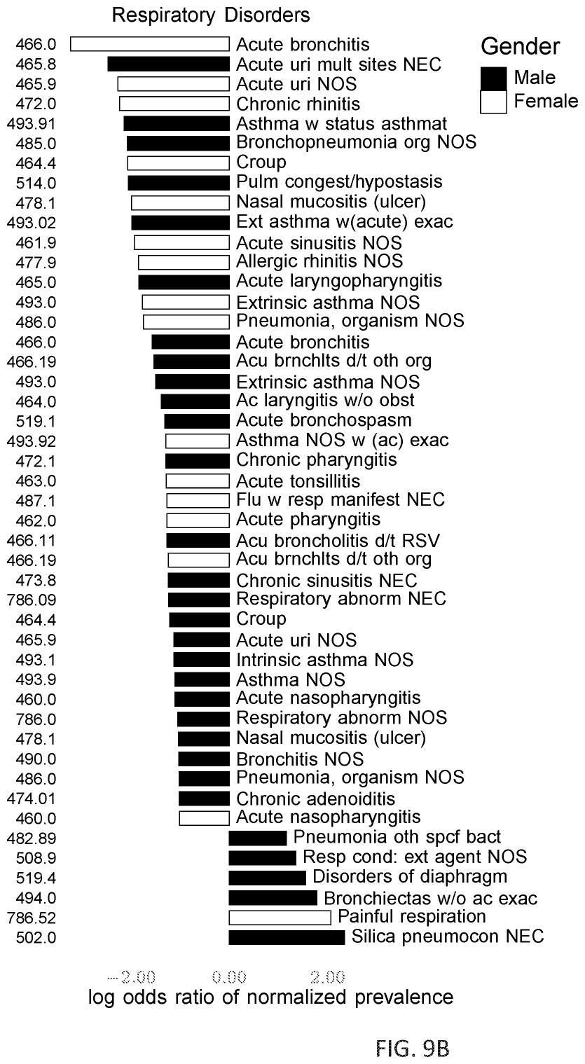

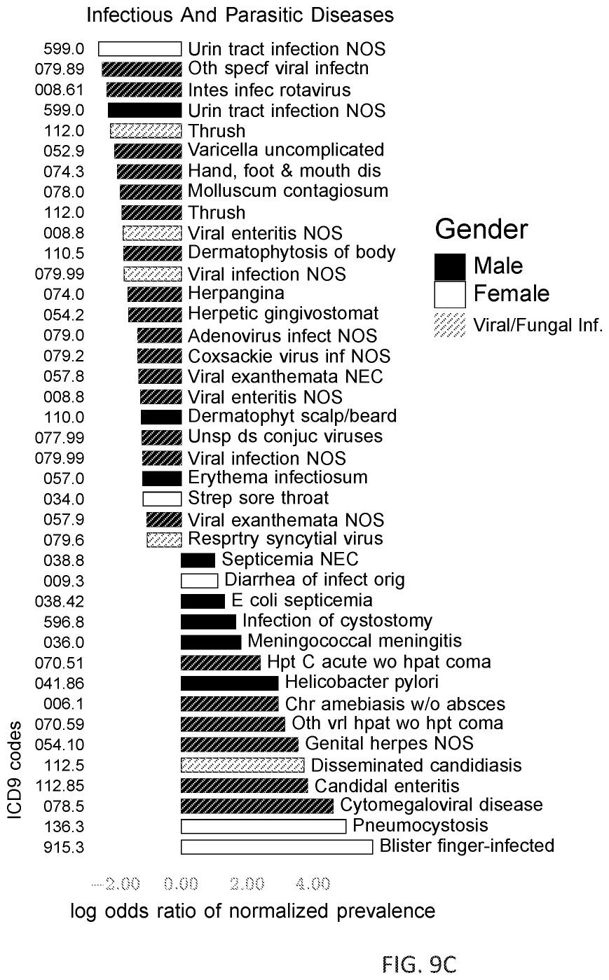

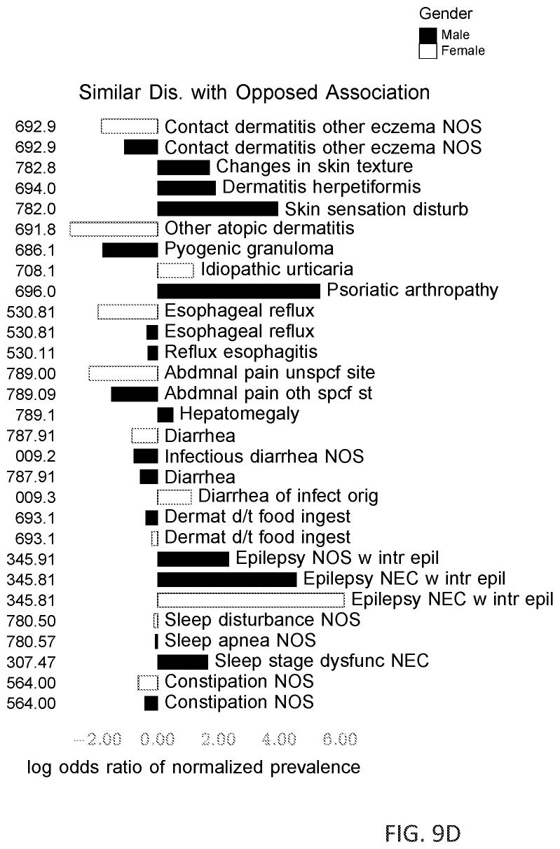

FIGS. 9A-D illustrates details of co-morbidity patterns at age <3 years for immunologic (FIG. 9A), respiratory (FIG. 9B), infections (FIG. 9C), and disorders with similar pathobiology manifesting opposing association with autism (FIG. 9D).

Despite reports, and with distinct prevalence patterns discernible between the treatment and the control populations (See FIG. 5C), the prior art lacks a risk estimator that makes reliable predictions for individual patients. The risk estimator disclosed herein provides a principled framework to make predictions based on models of statistically curated patterns of diagnostic code sequences automatically learned from sufficiently large databases of electronic health records (EHR). In some embodiments, the risk estimator may achieve an out-of-sample AUC exceeding 80% for either gender, for ages between 2 and 2.5 years (See FIG. 6 and FIG. 7A).

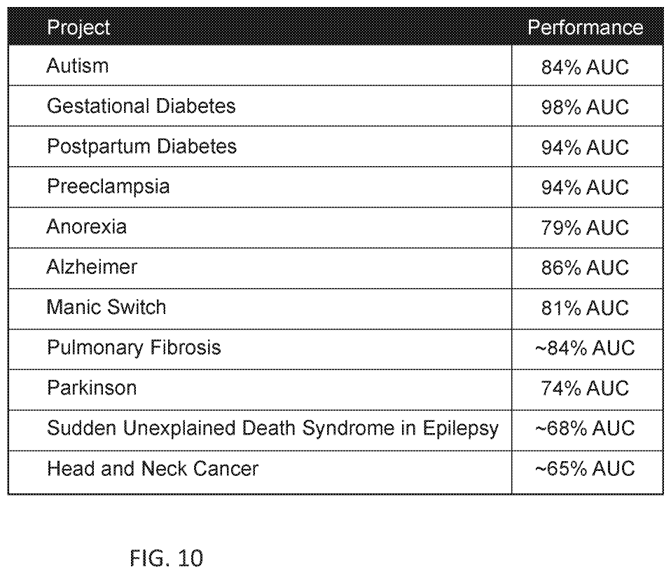

FIG. 10 illustrates example receiver operating characteristic (AUC) sensitivity ratings in accordance with one or more embodiments of the present disclosure. For example, varying levels of accuracy can be shown across varying types of disorders or diseases. Additional disorders, or diseases, that may be predicted include, but are not limited to Postpartum Diabetes, Preeclampsia, Anorexia, Alzheimer's, Manic Switch, Pulmonary Fibrosis, Parkinson's, Sudden Unexplained Death Syndrome in Epilepsy, and Head and Neck Cancer, for example. The algorithm implemented by the systems and methods described herein may further provide an accurate prediction of comorbid risks in these additional disorders. For example, the stochastic learning algorithm may be utilized to predict disorders accurately using relevant diagnosis codes (e.g., ICD9 or ICD10 codes) and certain patient-specific data as described.

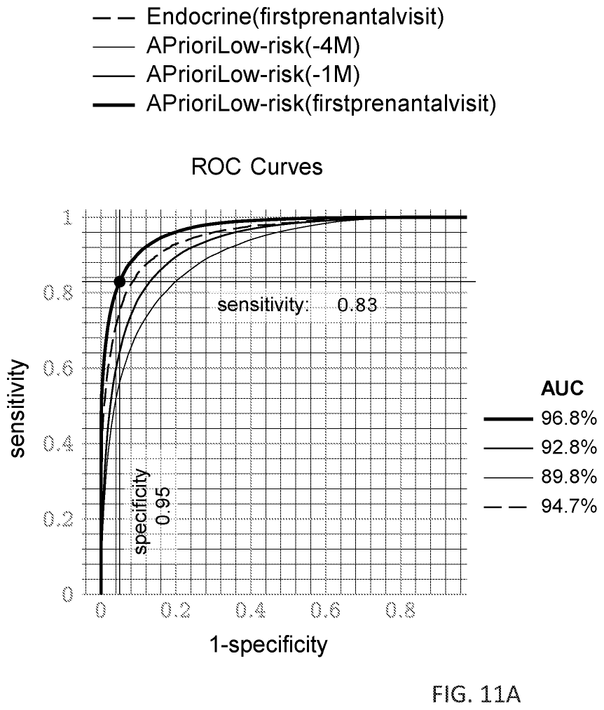

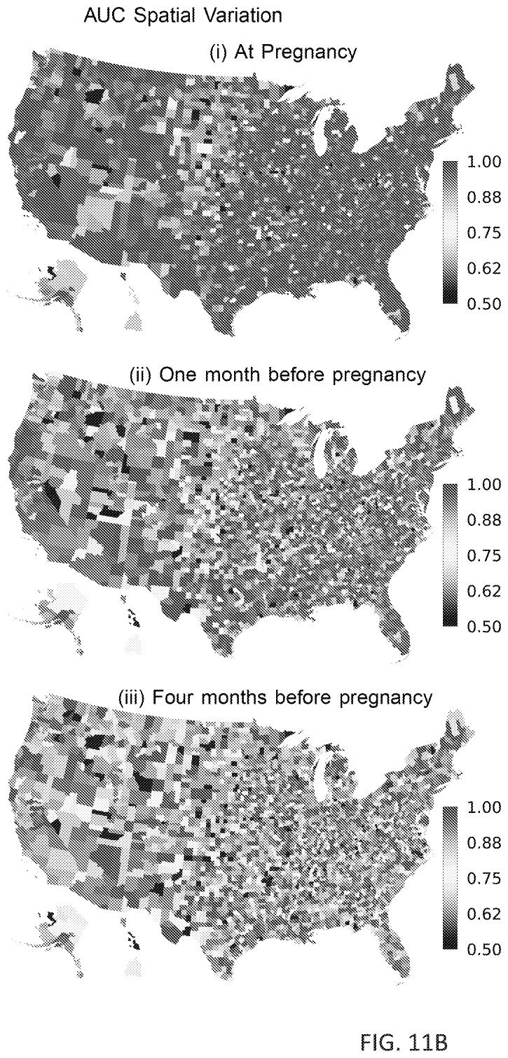

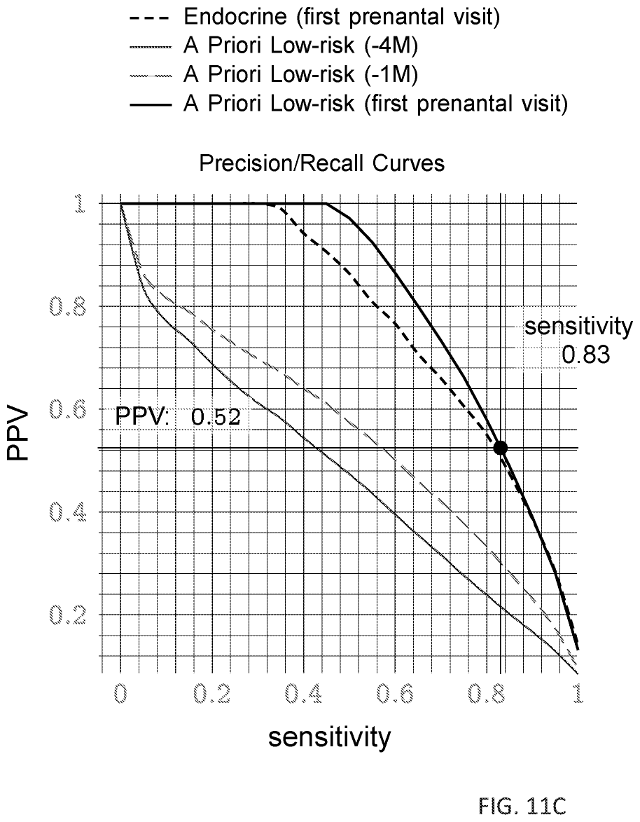

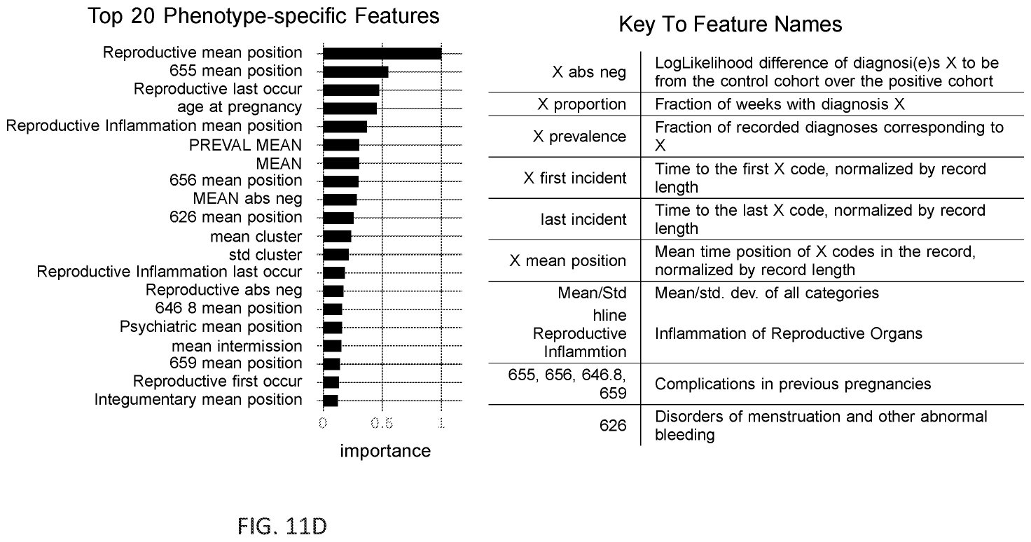

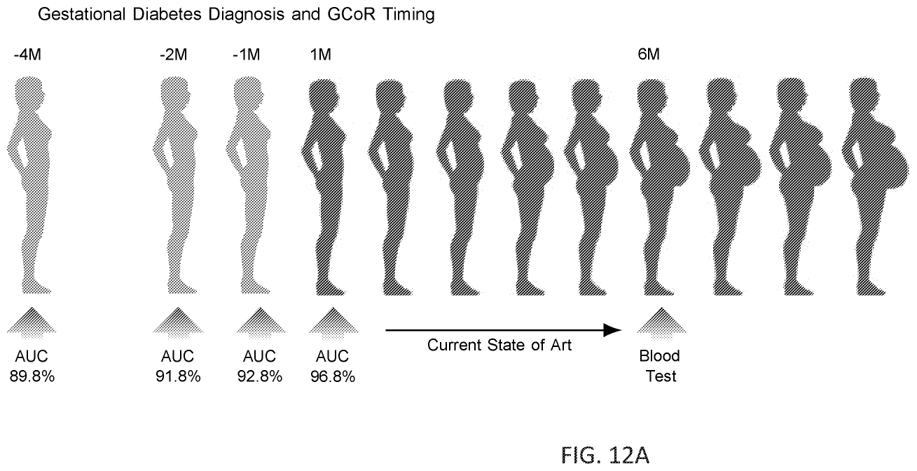

FIGS. 11A-11D illustrate example prediction performance. For example, FIG. 11A illustrates AUC with respect to sensitivity vs. specificity. FIG. 11A, for example, shows AUC exceeding 96% at the first pre-natal visit for patients without past encounters/diagnoses that unambiguously increase risk of gestational diabetes (a priori low-risk). High AUCs are shown for predictions made one and four months before pregnancy for the same cohort. FIG. 11B illustrates performance is stable across US counties, and degrades as predictions are made earlier, falling under 90% at four months to pregnancy. FIG. 11C illustrates precision-recall or the PPV vs. sensitivity curves, which in combination with FIG. 11A, shows 95% specificity, 83% sensitivity, and PPV exceeding 52%. FIG. 11D illustrates relative importance of top 20 features in the predictor, which reveals that reproductive disorders dominate the scale, followed by complications in previous pregnancies, and inflammation in reproductive organs.

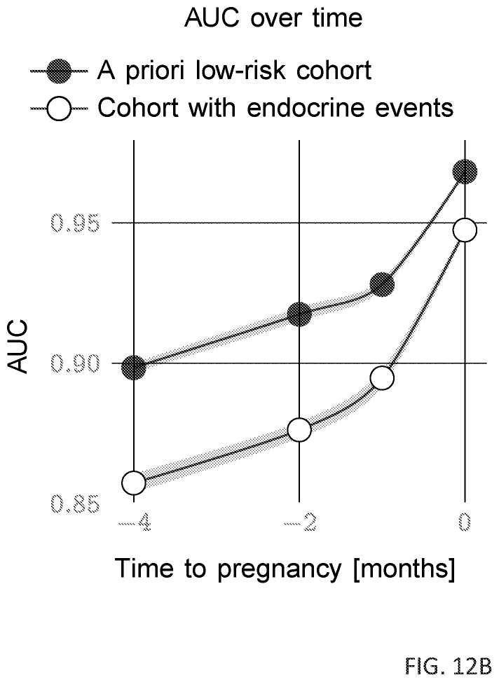

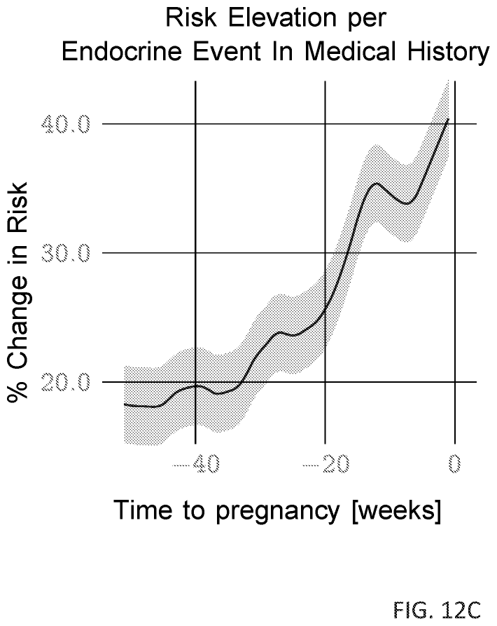

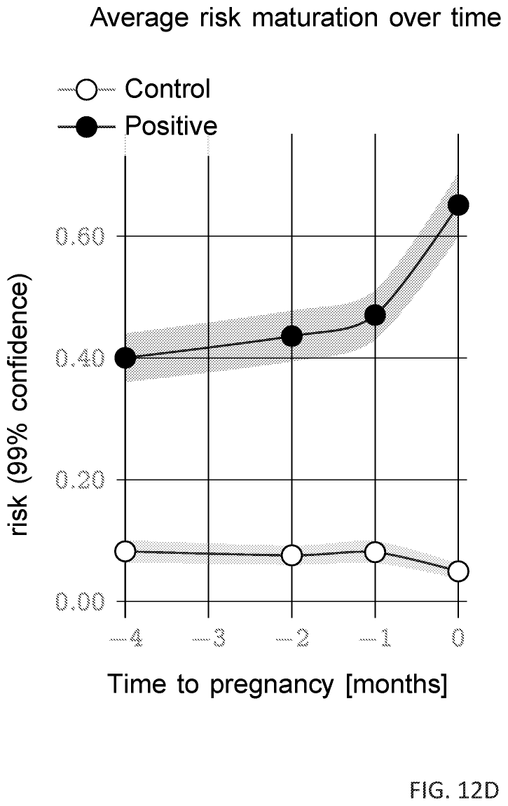

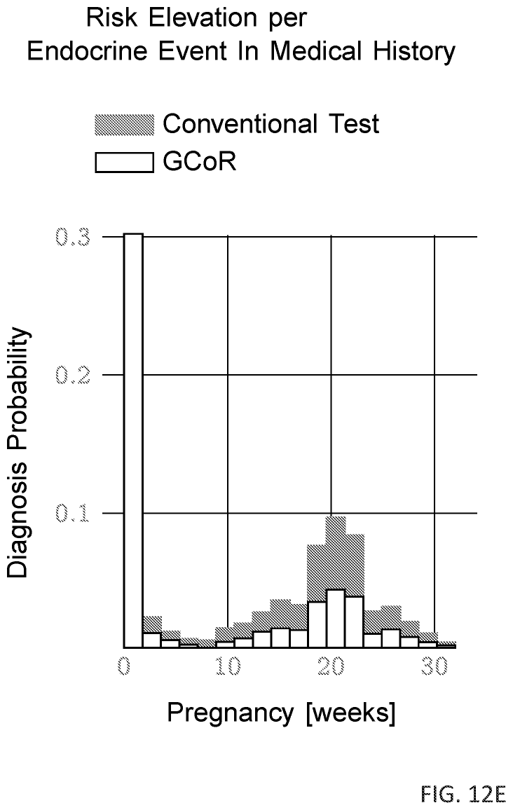

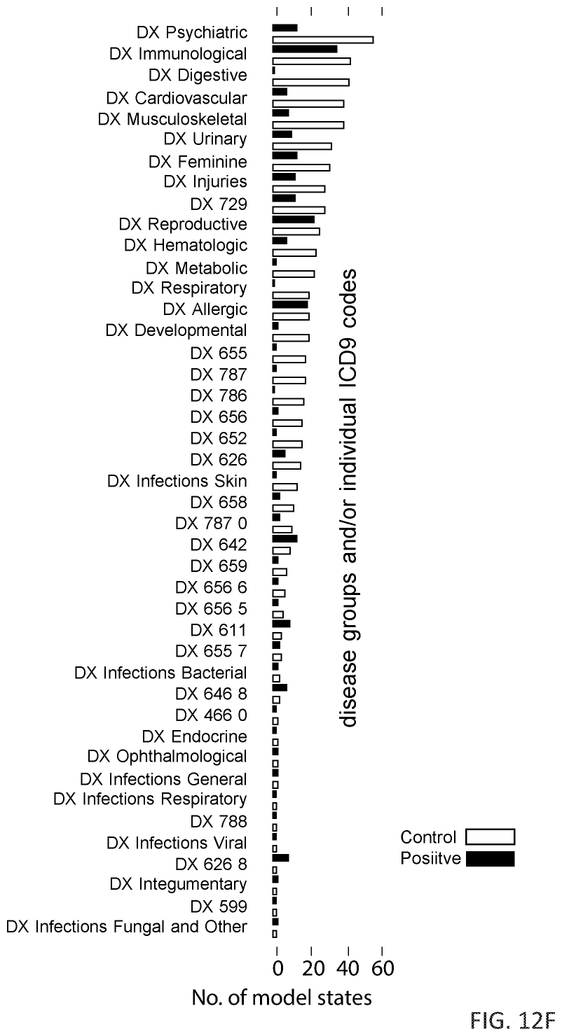

FIGS. 12A-12F illustrate example predictive performance, risk variation over time, and model complexity. FIG. 12A illustrates relative timings of the proposed prediction compared to that of current laboratory evaluation of GDM status from blood glucose levels. FIG. 12B shows the AUC variation over time. FIG. 12C illustrates the elevation of GDM risk from a single endocrinal diagnosis/event. The exponential elevation of risk with time suggests delaying pregnancy by 5-6 months on encountering such events might reduce risk of GDM. FIG. 12D illustrates the significant separation of raw estimated risk on average from four months before pregnancy to the pre-natal visit. FIG. 12E illustrates a comparison of the distribution of predicted GDM diagnoses over time against the time distribution of first DM codes as they appear over the course of pregnancy in the patient database. FIG. 12F illustrates a comparison of the complexity of the HMM models inferred from different diagnostic categories between the control and the positive cohorts, suggesting that the complexity in the case of the positive cohort is reduced on average, which suggests that event sequences are relatively more random with reduced long-term dependencies.

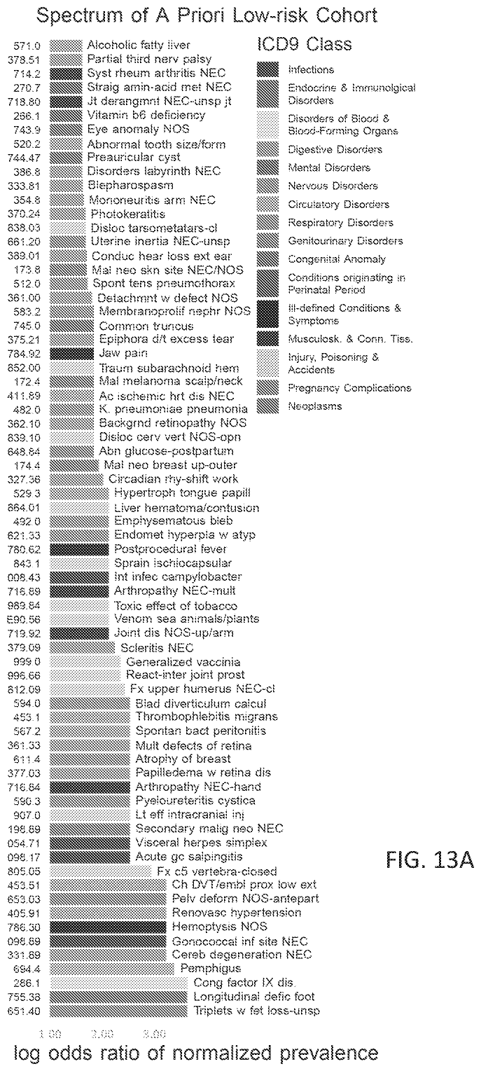

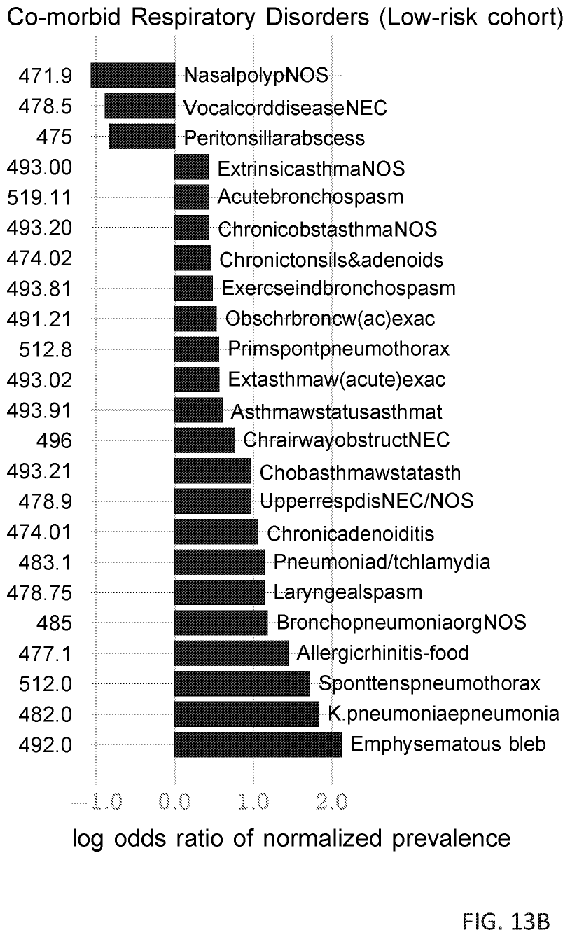

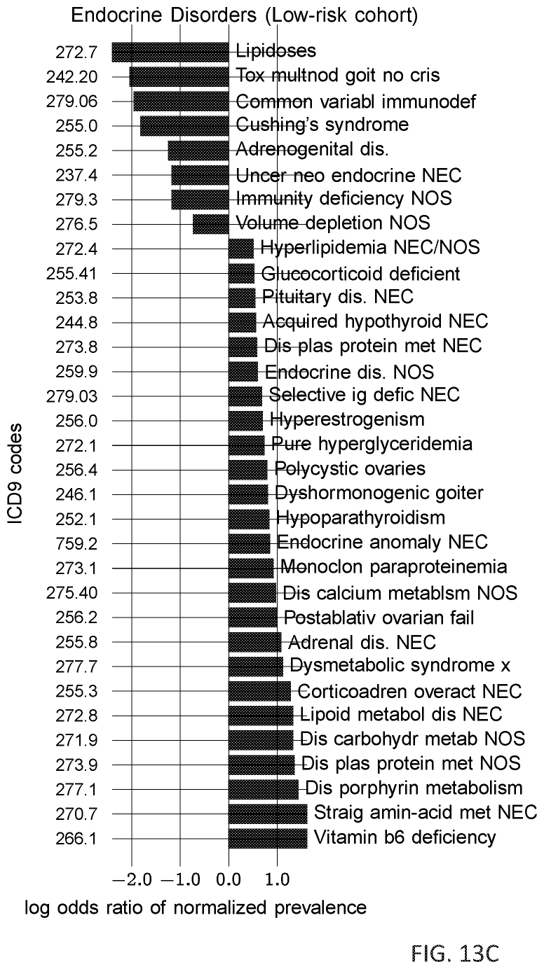

FIGS. 13A-13C illustrate example co-morbidity spectra. For example, FIG. 13A illustrates a log-odds ratio of the occurrence probability of individual diagnoses in the true positive vs the true negative sets of patients lacking obvious risk-elevating disorders/encounters, evaluated at the first pre-natal visit. FIGS. 13B and 13C illustrate the spectra of top individual codes pertaining to respiratory and non-obvious endocrinal events/disorders disambiguating true positive and true negative patients.

Some embodiments involve the use of one or more electronic processing or computing devices. As used herein, the terms “processor” and “computer” and related terms, e.g., “processing device,” “computing device,” and “controller” are not limited to just those integrated circuits referred to in the art as a computer, but broadly refers to a processor, a processing device, a controller, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processing (DSP) device, an application specific integrated circuit (ASIC), and other programmable circuits or processing devices capable of executing the functions described herein, and these terms are used interchangeably herein. The above embodiments are examples only, and thus are not intended to limit in any way the definition or meaning of the terms processor, processing device, and related terms.

In the embodiments described herein, memory may include, but is not limited to, a non-transitory computer-readable medium, such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROMs, DVDs, and any other digital source such as a network or the Internet, as well as yet to be developed digital means, with the sole exception being a transitory, propagating signal. Alternatively, a floppy disk, a compact disc-read only memory (CD-ROM), a magneto-optical disk (MOD), a digital versatile disc (DVD), or any other computer-based device implemented in any method or technology for short-term and long-term storage of information, such as, computer-readable instructions, data structures, program modules and sub-modules, or other data may also be used. Therefore, the methods described herein may be encoded as executable instructions, e.g., “software” and “firmware,” embodied in a non-transitory computer-readable medium. Further, as used herein, the terms “software” and “firmware” are interchangeable, and include any computer program stored in memory for execution by personal computers, workstations, clients and servers. Such instructions, when executed by a processor, cause the processor to perform at least a portion of the methods described herein. The systems and methods described herein are not limited to the specific embodiments described herein, but rather, components of the systems and/or steps of the methods may be utilized independently and separately from other components and/or steps described herein.

As will be appreciated based upon the disclosure herein, the above-described aspects of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving medium, such as the Internet or other communication network or link. The article of manufacture containing the computer code may be made and/or used by executing the code directly from one medium, by copying the code from one medium to another medium, or by transmitting the code over a network.

Embodiments of the disclosure may be described in the general context of computer-executable instructions, such as program modules, executed by one or more computers or other devices. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the FIGs and described herein. Other embodiments of the disclosure may include different computer-executable instructions or components having more or less functionality than illustrated and described herein. Aspects of the disclosure may also be practiced in distributed computing environments where tasks are performed by remote processing devices that are linked through a communications network. In a distributed computing environment, program modules may be located in both local and remote computer storage media including memory storage devices.

The computer systems, computing devices, and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include, but are not limited to: patient data. ML outputs may include, but are not limited to patient data and diagnostic data. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs, and utilize the predictive function to generate ML outputs based on data inputs. The example inputs and example outputs of the training data may include any of the data inputs or ML outputs described above. For example, a ML module may receive training data comprising patient data, generate a model that maps patient data to diagnostic data and generate a ML output comprising a prediction for subsequently received data inputs including new patient data.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In one aspect, a ML module receives unlabeled data comprising patient data, and the ML module employs an unsupervised learning method such as “clustering” to identify patterns and organize the unlabeled data into meaningful groups. The newly organized data may be used, for example, to extract further information about a disease or disorder diagnosis.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate a ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, a ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

The systems and methods described herein are not limited to the specific embodiments described herein, but rather, components of the systems and/or steps of the methods may be utilized independently and separately from other components and/or steps described herein.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to disclose various embodiments, which include the best mode, to enable any person skilled in the art to practice those embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### TABLES

TABLE I
Disease Categories (A few ICD9 codes shown.
See supplementary text for complete list)

| Category‡ | Description | Examples of ICD9 Codes |
|---|---|---|
| Hematologic | Diseases Of The Blood And Blood- | 286.9 286.6 283.19 283.9 283.1 |
| | Forming Organs | 284.0 284.09 284 284.01 |
| Metabolic | Metabolic Disorders (Non- | 273.4 270 270.3 712.11 712.12 |
| | overlapping with respiratory, | 712.14 712.18 712.30 712.37 |
| | digestive and immunological | 712.36 |
| | conditions) | |
| Cardiovascular | Diseases Of Arteries, Arterioles, | 442.89 441.6 442.82 442.83 |
| | And Capillaries | 441.03 441.02 441.00 442 414.11 |
| | | 447.70 447.71 |
| Reproductive | Diseases Of The Genitourinary | 611.79 611.71 611.89 611.81 |
| | System | 676.64 611 676.60 611.6 611.4 |
| | | 611.3 611.2 |
| Endocrine | Disorders Of Thyroid and other | 244 244.9 244.2 255.41 255.5 |
| | Endocrine Glands | 255.4 259.51 255 259.4 255.11 |
| | | 242.2 |
| Integumentary | Diseases Of Skin and Subcutaneous | 706.0 706.1 704.00 704.02 |
| | Tissue | 704.09 680.9 680.1 680.5 680.7 |
| | | 680.6 680 |
| Infectious | Diseases Caused By Pathogens | 487.8 488.12 488.0 488.01 487.0 |
| | | 487.1 488.09 464.4 466 466.11 |
| | | 466.1 |
| Respiratory | Diseases Of The Respiratory System | 516.31 516.30 516.32 516.35 |
| | (non-overlapping with Infectious) | 516.37 516.36 516.8 516.0 277.0 |
| | | 277.00 277.01 |
| Digestive | Diseases Of The Digestive System | 540.0 540.1 541.0 542 540 541 |
| | | 543. 562.03 562.01 562.00 |
| | | 562.10 |
| Immunologic | Diseases related to dys-regulation | 580.81 580.89 580.0 580.8 461 |
| | of the Immune system | 461.8 461.0 477.9 477.2 477 |
| | | 477.8 |
| Ophthalmologic | Disorders Of The Eye and Adnexa | 362.8 362.9 362.6 362.1 362.3 |
| | | 362.18 362.17 362.13 362.11 |
| | | 363.33 363.32 |
| Otic | Diseases Of The Ear And Adnexa | 381.51 381.50 381.81 381.89 |
| | | 381.61 381.62 381 381.7 385.82 |
| | | 383.32 380.30 |
| Musculoskeletal | Diseases Of The Eye And Mastoid | 756.52 756.53 733.02 733.0 |
| | Process | 733.09 737.43 737.41 737.20 |
| | | 737.29 737.4 737.2 |
| Developmental | Congenital anomalies (Non- | 755.55 743.45 743.11 743.10 |
| | overlapping with musculoskeletal) | 743.00 743.03 743.44 743.22 |
| | | 743.20 743.21 758.4 |
| Nutrition | Nutritional development | 783.0 783.21 783.3 783.40 |
| | | 783.42 783.7 783.9 |

‡Categories inferred to be important for risk modulation are highlighted.

TABLE II
Engineered Features (Total Count: 93)

| | | No. of |
|---|---|---|
| Feature Type‡ | Description | Features |
| [Disease Category]_{/}_\ | Likelihood Defect | 15 |
| [Disease Category]ₚᵣₒₚₒᵣₜᵢₒₙ | Occurrences in the encoded sequence/length of the | 15 |
| | sequence | |
| [Disease Category]ₛₜᵣₑₐₖ | Length of the longest subsequence of adjacent | 15 |
| | occurrences | |
| [Disease Category]ᵢₙₜₑᵣₘᵢₛₛᵢₒₙ | Length of the longest subsequence of adjacent empty | 15 |
| | weeks | |
| [Disease Category]_{prevalence} | Occurrences in the encoded sequence/Total Number | 15 |
| | of diagnostic codes in the mapped sequence | |
| [Disease Category]_{dynamics} | Occurrences in the second half of the | 15 |
| | sequence/Occurrences in the first half of the | |
| | sequence | |
| Feature Mean, Feature | Mean, Variance, Range of the [Disease Category] | 3 |
| Variance, Feature Range | values | |

‡Disease categories are described in Table I

TABLE III
Patient Counts In De-identified Data

| | Distinct Patients | | | |
|---|---|---|---|---|
| | Truven | | UCM | |
| | 115805687 | | 69484 | |
| | Male | Female | Male | Female |
| ASD Diagnosis | 12440 | 3245 | 418 | 97 |
| Count‡ | | | | |
| Control Count‡ | 2056339 | 1916732 | 82578 | 24778 |
| AUC at 100 weeks | 83.3% | 81.0% | 85.2% | 83.3% |

‡Cohort sizes are smaller than the total number of distinct patients due to the following exclusion criteria: 1) age between 0-5 years, and 2) at least one diagnostic code within our disease categories within the first 30 weeks of life.

TABLE IV
Target Codes: Description of ICD9 Codes(s)
Used To Identify Gestational Diabetes

| ICD9 Code* | Description |
|---|---|
| 648.0 | Diabetes mellitus complicating pregnancy childbirth or the |
| 648.00 | puerperium |
| 648.01 | Diabetes mellitus of mother, complicating pregnancy, |
| | childbirth, or the puerperium, |
| | unspecified as to episode of care or not applicable |
| 648.03 | Diabetes mellitus of mother, complicating pregnancy, |
| | childbirth, or the puerperium, |
| | delivered, with or without mention of antepartum condition |
| | Diabetes mellitus of mother, complicating pregnancy, |
| | childbirth, or the puerperium, |
| | antepartum condition or complication |
| 648.8 | Abnormal glucose in pregnancy-unspecified |
| 648.83 | Abnormal glucose antepartum |

*ICD10 codes are mapped to their closest ICD9 counterparts using GEMS mapping

TABLE V
Inclusion/Exclusion, Positive/Control Criteria & Cohort Definitions

| | Definitions |
|---|---|
| Inclusion/Exclusion | ICD9 code for pregnancy (V22.0, V22.1) |
| Criteria | observed |
| | Patients exist in database for at least 52 weeks |
| | before pregnancy |
| Positive & Control | Positive Cohort: Patients with at least one |
| Cohorts | target code (Tab. I) |
| | Control Cohort: Patients lacking any target |
| | code within 32 weeks of pregnancy record (first |
| | appearance of V22.0, V22.1) |
| General cohort | Patients who may include past diabetes and |
| | other risk-increasing historical |
| | diagnoses |
| A Priori Low-risk cohort | Patients with prexisting diabetes and other risk- |
| | increasing diagnoses excluded‡ |
| Endocrine cohort | Patients experiencing endocrinal disorders in |
| | the year before pregnancy |

‡See Tab. VI for list of diagnoses considered to be risk-increasing

TABLE VI
Cohort Sizes

| | | | | age [yr] | | |
|---|---|---|---|---|---|---|
| cohort | ^{η} | ^{η}pos | ^{η}control | 16-21 | 21-35 | >35 |
| A Priori Low-risk§ | 548,784 | 4,221 | 644,563 | 6,731 | 564,656 | 77,397 |
| General§ | 570,417 | 4,655 | 665,762 | 6,826 | 582,793 | 80,798 |
| Endocrine§ | 104,946 | 1,335 | 103,611 | 550 | 90,340 | 14,056 |

§See Tab. V for cohort definitions

TABLE VII
Summarized Performance for Different
Cohorts & Prediction Timepoints

| | | First | | | |
|---|---|---|---|---|---|
| | | Prenantal | 1 month | 2 months | 4 months |
| cohort | metric | Visit | earlier | earlier | earlier |
| a priori | AUC | 96.87 | 92.75 | 91.82 | 89.97 |
| low risk | sensitivity‡ | 85.12 | 68.47 | 65.80 | 59.87 |
| | PPV‡ | 53.85 | 48.43 | 47.44 | 45.09 |
| general | AUC | 95.42 | 89.24 | 88.06 | 86.08 |
| | sensitivity‡ | 78.80 | 58.60 | 52.36 | 44.02 |
| | PPV‡ | 53.38 | 44.60 | 42.59 | 39.12 |
| high risk | AUC | 94.83 | 89.31 | 87.51 | 85.80 |
| | sensitivity‡ | 77.99 | 58.71 | 55.29 | 49.73 |
| | PPV‡ | 51.69 | 44.67 | 43.13 | 40.55 |

‡Calculated at 95% specificity and 7.6% prevalence

Select 50% of the patients for training our models in each case, holding back the remaining for out-of-sample evaluation.

TABLE VIII
Number of Codes Per Patient In Training

| cohort | class | mean | median | [symbol] |
|---|---|---|---|---|
| a priori low-risk | control | 9.60 | 6.00 | 10.33 |
| a priori low-risk | positive | 8.57 | 4.00 | 9.54 |
| general | control | 18.13 | 14.00 | 15.75 |
| general | positive | 11.60 | 6.00 | 11.10 |
| endocrine | control | 12.97 | 9.00 | 13.46 |
| endocrine | positive | 10.83 | 4.00 | 11.24 |

TABLE IX
High Risk Pre-conditions Excluded in The A Priori Low-risk Cohort

| ICD9 code | Description |
|---|---|
| code | description |
| 251.5 | Abnormality of secretion of gastrin |
| 251.4 | Abnormality of secretion of glucagon |
| 251.3 | Postsurgical hypoinsulinemia |
| 251.2 | Hypoglycemia, unspecified |
| 251.1 | Other specified hypoglycemia |
| 251.0 | Hypoglycemic coma |
| 249.01 | Secondary diabetes mellitus without mention of complication, uncontrolled |
| 249.00 | Secondary diabetes mellitus without mention of complication, not stated as |
| | uncontrolled, or unspecified |
| 251.9 | Unspecified disorder of pancreatic internal secretion |
| 251.8 | Other specified disorders of pancreatic internal secretion |
| 278.01 | Morbid obesity |
| 278.01 | Obesity, unspecified |
| 278.02 | Overweight |
| 249.81 | Secondary diabetes mellitus with other specified manifestations, uncontrolled |
| 249.80 | Secondary diabetes mellitus with other specified manifestations, not stated as |
| | uncontrolled, or unspecified |
| 250.13 | Diabetes with ketoacidosis, type I [juvenile type], uncontrolled |
| 250.21 | Diabetes with hyperosmolarity, type I [juvenile type], not stated as uncontrolled |
| 250.20 | Diabetes with hyperosmolarity, type II or unspecified type, not stated as |
| | uncontrolled |
| 250.23 | Diabetes with hyperosmolarity, type I [juvenile type], uncontrolled |
| 250.22 | Diabetes with hyperosmolarity, type II or unspecified type, uncontrolled |
| 250.43 | Diabetes with renal manifestations, type I [juvenile type], uncontrolled |
| 250.42 | Diabetes with renal manifestations, type II or unspecified type, uncontrolled |
| 250.41 | Diabetes with renal manifestations, type I [juvenile type], not stated as |
| | uncontrolled |
| 250.40 | Diabetes with renal manifestations, type II or unspecified type, not stated as |
| | uncontrolled |
| 250.03 | Diabetes mellitus without mention of complication, type I [juvenile type], |
| | uncontrolled |
| 250.02 | Diabetes mellitus without mention of complication, type II or unspecified type, |
| | uncontrolled |
| 250.01 | Diabetes mellitus without mention of complication, type I [juvenile type], not |
| | stated as uncontrolled |
| 250.00 | Diabetes mellitus without mention of complication, type II or unspecified type, |
| | not stated as uncontrolled |
| 249.50 | Secondary diabetes mellitus with ophthalmic manifestations, not stated as |
| | uncontrolled, or unspecified |
| 249.60 | Secondary diabetes mellitus with neurological manifestations, not stated as |
| | uncontrolled, or unspecified |
| 250.83 | Diabetes with other specified manifestations, type I [juvenile type], uncontrolled |
| 250.82 | Diabetes with other specified manifestations, type II or unspecified type, |
| | uncontrolled |
| 250.81 | Diabetes with other specified manifestations, type I [juvenile type], not stated as |
| | uncontrolled |
| 250.80 | Diabetes with other specified manifestations, type II or unspecified type, not |
| | stated as uncontrolled |
| 278.1 | Localized adiposity |
| 249.70 | Secondary diabetes mellitus with peripheral circulatory disorders, not stated as |
| | uncontrolled, or unspecified |
| 250.60 | Diabetes with neurological manifestations, type II or unspecified type, not stated |
| | as uncontrolled |
| 250.63 | Diabetes with neurological manifestations, type I [juvenile type], uncontrolled |
| 250.62 | Diabetes with neurological manifestations, type II or unspecified type, |
| | uncontrolled |
| 278.8 | Other hyperalimentation |
| 249.21 | Secondary diabetes mellitus with hyperosmolarity, uncontrolled |
| 249.30 | Secondary diabetes mellitus with other coma, not stated as uncontrolled, or |
| | unspecified |
| 249.31 | Secondary diabetes mellitus with other coma, uncontrolled |
| 278.2 | Hypervitaminosis A |
| 278.3 | Hypercarotinemia |
| 278.4 | Hypervitaminosis D |
| 249.10 | Secondary diabetes mellitus with ketoacidosis, not stated as uncontrolled, or |
| | unspecified |
| 249.11 | Secondary diabetes mellitus with ketoacidosis, uncontrolled |
| 249.20 | Secondary diabetes mellitus with hyperosmolarity, not stated as uncontrolled, or |
| | unspecified |
| 249.90 | Secondary diabetes mellitus with unspecified complication, not stated as |
| | uncontrolled, or unspecified |
| 249.91 | Secondary diabetes mellitus with unspecified complication, uncontrolled |
| 250.11 | Diabetes with ketoacidosis, type I [juvenile type], not stated as uncontrolled |
| 249.61 | Secondary diabetes mellitus with neurological manifestations, uncontrolled |
| 250.32 | Diabetes with other coma, type II or unspecified type, uncontrolled |
| 250.33 | Diabetes with other coma, type I [juvenile type], uncontrolled |
| 250.30 | Diabetes with other coma, type II or unspecified type, not stated as uncontrolled |
| 250.31 | Diabetes with other coma, type I [juvenile type], not stated as uncontrolled |
| 250.61 | Diabetes with neurological manifestations, type I [juvenile type], not stated as |
| | uncontrolled |
| 250.10 | Diabetes with ketoacidosis, type II or unspecified type, not stated as |
| | uncontrolled |
| 249.51 | Secondary diabetes mellitus with ophthalmic manifestations, uncontrolled |
| 250.12 | Diabetes with ketoacidosis, type II or unspecified type, uncontrolled |
| 249.71 | Secondary diabetes mellitus with peripheral circulatory disorders, uncontrolled |
| 249.41 | Secondary diabetes mellitus with renal manifestations, uncontrolled |
| 249.40 | Secondary diabetes mellitus with renal manifestations, not stated as |
| | uncontrolled, or unspecified |
| 250.90 | Diabetes with unspecified complication, type II or unspecified type, not stated as |
| | uncontrolled |
| 250.91 | Diabetes with unspecified complication, type I [juvenile type], not stated as |
| | uncontrolled |
| 250.92 | Diabetes with unspecified complication, type II or unspecified type, uncontrolled |
| 250.93 | Diabetes with unspecified complication, type I [juvenile type], uncontrolled |
| 250.50 | Diabetes with ophthalmic manifestations, type II or unspecified type, not stated |
| | as uncontrolled |
| 250.51 | Diabetes with ophthalmic manifestations, type I [juvenile type], not stated as |
| | uncontrolled |
| 250.52 | Diabetes with ophthalmic manifestations, type II or unspecified type, |
| | uncontrolled |
| 250.53 | Diabetes with ophthalmic manifestations, type I [juvenile type], uncontrolled |
| 250.72 | Diabetes with peripheral circulatory disorders, type II or unspecified type, |
| | uncontrolled |
| 250.73 | Diabetes with peripheral circulatory disorders, type I [juvenile type], |
| | uncontrolled |
| 250.70 | Diabetes with peripheral circulatory disorders, type II or unspecified type, not |
| | stated as uncontrolled |
| 250.71 | Diabetes with peripheral circulatory disorders, type I [juvenile type], not stated |
| | as uncontrolled |

1. A method for estimating risk of disease diagnosis by a computing device, the method comprising:
retrieving unprocessed raw data associated with a plurality of patients;
building a model relating elements of the unprocessed raw data, wherein building the model further comprises:
partitioning a human disease spectrum into one or more categories;
generating one or more categorical time series based on the unprocessed raw data;
constructing a set of statistical models representing the one or more categories;
determining, for each of the one or more categories, a sequence likelihood defect (SLD) value;
training a tree-based classifier based on one or more features extracted from the unprocessed raw data;
assigning a weight to each of the one or more features based at least in part on the SLD values;
constructing an estimator based on the statistical model and the weighted one or more features; and
validating the estimator; and
receiving patient-specific data associated with at least one patient; and
predicting a likelihood of a disease diagnosis for the at least one patient using the model based upon the received patient-specific data.

2. The method of claim 1, wherein the method further comprises:
generating one or more intervention possibilities based on the predicted likelihood.

3. The method of claim 1, wherein the unprocessed raw data is received from an insurance claims database, a health records database, or both.

4. The method of claim 1, wherein the unprocessed raw data consists essentially of records of diagnostic codes generated during past medical encounters of the plurality of patients.

5. The method of claim 1, wherein the set of statistical models are further constructed to represent genders, a treatment cohort, and a control cohort based on the unprocessed raw data.

6. The method of claim 1, wherein the disease diagnosis is an Autism Spectrum Diagnosis (ASD) diagnosis, a Pulmonary Fibrosis diagnosis, a Alzheimer's diagnosis or a Dementia diagnosis.

7. The method of claim 1, wherein the disease diagnosis is related to Autism Spectrum Diagnosis (ASD) and is at least one of the following: Angelman Syndrome, Fragile X Syndrome, Landau-Kleffner Syndrome, Prader-Willi Syndrome, Tardive Dyskinesia, and Williams Syndrome.

8. The method of claim 1, wherein the unprocessed raw data includes diagnostic history of at least some of the plurality of patients.

9. The method of claim 1, wherein the likelihood is predicted for different cohorts of the plurality of patients at different time-points.

10. The method of claim 1, wherein the likelihood provides one or more cues to other disorders misdiagnosed as a different disorder for the at least one patient.

11. The method of claim 1, wherein the unprocessed raw data includes one or more individual diagnostic codes from prior doctor visits made by one or more of the plurality of patients.

12. The method of claim 1, wherein the patient-specific data includes one or more sequences of diagnostic codes from past doctor's visits by the at least one patient.

13. The method of claim 1, wherein the likelihood is predicted without any new blood work for the at least one patient.

14. The method of claim 1, wherein the model further comprises a representation of each patient of the plurality of patients by a mapped trinary series to infer one or more population-level models.

15. The method of claim 14, wherein each of the mapped trinary series is stratified by gender, disease-category, and disease diagnosis status.

16. The method of claim 14, wherein each of the inferred population-level models includes a modeling of treatment and control for each gender in each disease category separately.

17. A non-transitory computer-readable medium comprising instructions for estimating risk of disease diagnosis, the instructions, when executed by a processor, implement:
retrieving unprocessed raw data associated with a plurality of patients;
building a model relating elements of the unprocessed raw data, wherein building the model further comprises:
partitioning a human disease spectrum into one or more categories;
generating one or more categorical time series based on the unprocessed raw data;
constructing a set of statistical models representing the one or more categories;
determining, for each of the one or more categories, a sequence likelihood defect (SLD) value;
training a tree-based classifier based on one or more features extracted from the unprocessed raw data;
assigning a weight to each of the one or more features based at least in part on the SLD values;
constructing an estimator based on the statistical model and the weighted one or more features; and
validating the estimator; and
receiving patient-specific data associated with at least one patient; and
predicting a likelihood of a disease diagnosis for the at least one patient using the model based upon the received patient-specific data.

18. The non-transitory computer-readable medium of claim 17, wherein the model further comprises a representation of each patient of the plurality of patients by a mapped trinary series to infer one or more population-level models, each of the mapped trinary series is stratified by gender, disease-category, and disease diagnosis status, and each of the inferred population-level models includes a modeling of treatment and control for each gender in each disease category separately.

19. An apparatus for estimating risk of disease diagnosis, the apparatus comprising at least one processor in communication with at least one memory device, wherein the at least one processor is programmed to:
retrieve unprocessed raw data associated with a plurality of patients;
build a model relating elements of the unprocessed raw data, wherein to build the model the processor is further programmed to:
partition a human disease spectrum into one or more categories;
generate one or more categorical time series based on the unprocessed raw data;
construct a set of statistical models representing the one or more categories;
determine, for each of the one or more categories, a sequence likelihood defect (SLD) value;
train a tree-based classifier based on one or more features extracted from the unprocessed raw data;
assign a weight to each of the one or more features based at least in part on the SLD values;
construct an estimator based on the statistical model and the weighted one or more features; and
validate the estimator; and
receive patient-specific data associated with at least one patient; and
predict a likelihood of a disease diagnosis for the at least one patient using the model based upon the received patient-specific data.

20. The apparatus of claim 19, wherein the at least one processor is programmed to:
generate one or more intervention possibilities based on the predicted likelihood.
